(19) United States
(12) Patent Application Publication
(10) Pub. No.: US 2006/0069316 A1
(43) Pub. Date: Mar. 30, 2006

(54) Retracting devices

(75) Inventors: William Dorfman, Beverly Hills, CA (US); Robert Hayman, Los Angeles, CA (US); Eric P. Rose, Tarzana, CA (US); Dennis Schroeder, Hermosa Beach, CA (US); Steve Piorek, Los Angeles, CA (US); Nancy N. Quan, North Hills, CA (US); Stuart Karten, Venice, CA (US); Kenneth Rosenblood, Los Angeles, CA (US)

Correspondence Address: Michael Bergman;Bergman Kuta LLP, P.O. Box 400167, Cambridge, MA, 02140, US

(73) Assignee: Discus Dental Impressions, Inc., Culver City, CA (US)

(21) Appl. No.: 11/173,297

(22) Filed: Jun. 30, 2005

Related U.S. Application Data
(63) Continuation-in-part of application No. 29/220,642, filed on Jan. 4, 2005.
(63) Continuation-in-part of application No. 29/220,680, filed on Jan. 4, 2005.
(63) Continuation-in-part of application No. 29/220,679, filed on Jan. 4, 2005.
(63) Continuation-in-part of application No. 29/220,712, filed on Jan. 4, 2005.
(63) Continuation-in-part of application No. 29/232,670, filed on Jun. 22, 2005.
(63) Continuation-in-part of application No. 29/232,671, filed on Jun. 22, 2005.
(60) Provisional application No. 60/585,224, filed on Jul. 2, 2004.
(60) Provisional application No. 60/647,580, filed on Jan. 26, 2005.
(60) Provisional application No. 60/641,462, filed on Jan. 4, 2005.
(60) Provisional application No. 60/641,468, filed on Jan. 4, 2005.
(60) Provisional application No. 60/604,577, filed on Aug. 25, 2004.
(60) Provisional application No. 60/631,267, filed on Nov. 26, 2004.
(60) Provisional application No. 60/647,612, filed on Jan. 26, 2005.
(60) Provisional application No. 60/647,723, filed on Jan. 26, 2005.
(60) Provisional application No. 60/641,469, filed on Jan. 4, 2005.
(60) Provisional application No. 60/641,461, filed on Jan. 4, 2005.
(60) Provisional application No. 60/647,593, filed on Jan. 26, 2005.
(60) Provisional application No. 60/604,577, filed on Aug. 25, 2004.
(60) Provisional application No. 60/647,725, filed on Jan. 26, 2005.
(60) Provisional application No. 60/594,297, filed on Mar. 25, 2005.
(60) Provisional application No. 60/664,696, filed on Mar. 22, 2005.
(60) Provisional application No. 60/658,517, filed on Mar. 3, 2005.
(60) Provisional application No. 60/594,327, filed on Mar. 30, 2005.

(51) Int. Cl. A61B 1/32

(52) U.S. Cl. 600/237

(57) ABSTRACT

The present invention relates to a retracting device for retracting at least a portion of a user's mouth. The retracting device includes formations, which may be inter-engaging and/or non-inter-engaging with other dental tools or apparatus. The formations are adapted for repeatably positioning a subject's mouth with respect to a light system, and/or an imaging film, and/or a dental tray.

### CROSS-REFERENCE TO RELATED APPLICATIONS

This application claims the benefit of U.S. provisional applications Ser. No. 60/604,577, filed Aug. 25, 2004, entitled “Lip Retractors”; a copending U.S. patent application Ser. No. ______, filed Jun. 30, 2005, which claims priority from U.S. provisional patent applications Ser. No. 60/585,224, filed Jul. 2, 2004, entitled “Dental Light Devices With Phase Change Heat Sink”; 60/647,725, filed Jan. 26, 2005, entitled “Automatic Control for a Dental Whitening Lamp”; 60/658,517, filed Mar. 3, 2005, entitled “Apparatus and Method For Radiation Spectrum Shifting in Dentistry Application”; 60/641,469, filed Jan. 4, 2005, entitled “Lamp For Dentistry Applications”; 60/647,580, filed Jan. 26, 2005, entitled “Light Guide For Dental Whitening Lamp”; 60/641,468, filed Jan. 4, 2005, entitled “Light Guide For A Dental Whitening Lamp”; 60/641,462, filed Jan. 4, 2005, entitled “Boom Hinge For A Dental Lamp”; 60/647,723, filed Jan. 26, 2005, entitled “Boom Hinge For A Dental Lamp”; 60/641,461, filed Jan. 4, 2005, entitled “Support Structure For A Dental Lamp”; 60/647,612, filed Jan. 26, 2005, entitled “Light Path Apparatus For A Dental Lamp”; 60/647,593, filed Jan. 26, 2005, entitled “Support Structure For A Dental Lamp”; U.S. design patent applications Ser. No. 29/220,642, filed Jan. 4, 2005, entitled “Lamp For Dentistry Applications”; Ser. No. 29/220,680, filed Jan. 4, 2005, entitled “Light Guide For Dentistry Applications”; Ser. No. 29/220,679, filed Jan. 4, 2005, entitled “Power Pack For Dentistry Applications”; Ser. No. 29/220,712, filed Jan. 4, 2005, entitled “Support Structure For A Lamp For Dentistry”; U.S. provisional applications Ser. No. 60/604,577, filed Aug. 25, 2004, entitled “Lip Retractors”; 60/594,297, filed Mar. 25, 2005, entitled “Curing Light Having A Detachable Tip”; 60/631,267, filed Nov. 26, 2004, entitled “Curing Light Having A Reflector”; 60/594,327, filed on Mar. 30, 2005, entitled, “Curing Light”; and 60/664,696, filed Mar. 22, 2005, entitled “Curing Light Having A Detachable Tip”; the contents of all of which are hereby incorporated by reference.

The present application includes related to the claims of co-pending U.S. patent applications, Ser. No. 10/______, to be concurrently filed, entitled “Illumination System for Dentistry Applications”; Ser. No. 10/______, to be concurrently filed, entitled “Voice Alert System for Dentistry Applications”; and Ser. No. 10/______, to be concurrently filed, entitled “Support System for Dentistry”; Ser. No. 10/______, to be concurrently filed, entitled “Retracting Devices”; Ser. No. 10/______, to be concurrently filed, entitled “Light Guide for Dentistry Applications”; the contents of all of which are hereby incorporated by reference.

### FIELD OF THE INVENTION

The present invention relates to oral retracting devices in general. Specifically, the retracting devices are adapted for retracting portions of the mouth.

### BACKGROUND OF THE INVENTION

Mouth corner spreading devices, also known as cheek retractors or tongur cups, are well known in the art for spreading portions of the lips, which spread the cheeks, for examination and/or treatment by healthcare professionals. Exemplary mouth corner spreading devices include devices that spread a portion or several portions of the upper and lower lips using levers that are biased apart by an assistant, using flanges that cup and spread the lips, using devices that include metal resilient members, and using devices that have two retaining members for spreading two portions of the lips. However, there is still a need for a retracting device as described below for the advantages that are associated therewith.

### SUMMARY OF THE INVENTION

According to the present invention, there is provided a retracting device for retracting at least a portion of a user's mouth. The retracting device includes formations, which may be inter-engaging and/or non-inter-engaging with other dental tools or apparatus. The formations are adapted for repeatably positioning at least a portion of a subject's mouth with respect to a light system, and/or an imaging film, and/or a dental tray, and/or an apparatus adapted for aspiration, such as an aspirator, and/or a suction tube.

In one embodiment, a light system includes a spacer such as a light output port, an imaging device, a light guide or an examination device including inter-engaging formations for removably inter-engage as the spacer and the retracting device become apposed.

In another embodiment, a retracting device includes formations such as a dental tray adapted for repeatably positioning a subject's teeth with a treatment composition.

In a further embodiment, a retracting device includes formations such as an imaging device adapted for repeatably positioning a subject's teeth with respect to an imaging device, and/or a light source or imaging source.

In one embodiment of the present invention, the retracting device includes at least two channel retainers or flanges, at least one resilient member, and at least two formations such as wing-like members or flanges, wherein each of the channel retainers includes a race, an inside side wall, an outside side wall, and each of the wing-like members is spaced away from the attachment of the resilient member. Each of the wing-like members is adapted to fit into a slot in an output port, a light guide, an imaging device or an examination device such as a cone. In one aspect, each of the resilient members is attached to the inside side wall of two adjacent channel retainers by means of an adhesive or heat sealing, and includes two arches; and each of the wing-like flanges or members is attached to a channel retainer by means of an adhesive or heat sealing. In another aspect, each of the resilient members is integrally molded to the inside side wall of the two adjacent channel retainers and includes two arches; and each of the wing-like flanges or members is integrally molded to a channel retainer.

According to another embodiment of the invention, there is provided a retracting device for retracting a user's lips, including at least two channel retainers or flanges, at least one resilient member, at least two pads, and at least two formations such as wing-like flanges, wherein each channel retainer includes a race, an inside side wall, and an outside side wall; each resilient member is integrally molded or attached to the side walls of two adjacent channel retainers and includes at least one arch; each wing-like flange is integrally molded or attached to the outside wall of a channel retainer or flange at a location that is spaced away from the attachment of the resilient member; and the pad is attached or molded to the resilient member, for example, about the area of the arch.

According to yet another embodiment of the invention, there is provided a retracting device for retracting a user's lips, including at least two channel retainers, at least two formations such as wing-like flanges and a tongue retainer, the channel retainers being held in a spaced apart relationship by at least one resilient member, the wing-like flanges being integrally attached or molded to the channel retainers and the tongue retainer being attached to two of the channel retainers.

According to a further embodiment of the present invention, the lip retracting device includes at least four channel retainers or flanges, at least four resilient members, and at least two formations such as two wing-like members or flanges, wherein each channel retainer includes a race, an inside side wall, and an outside side wall; each resilient member is integrally molded or attached to two outside side walls of two adjacent channel retainers and includes an arch; and each wing-like member or flange is integrally molded or attached to a channel retainer or flange at a location that is spaced away from the attachment area of the resilient member. Each of the wing-like members is adapted to fit into a slot in an output port, a light guide, an imaging device or an examination device such as a cone.

According to yet a further embodiment of the present invention, there is provided a retracting device for retracting a user's lips, including four channel retainers, a plurality of resilient members, at least two formations such as wing-like members, at least two pads, and a tongue retainer, the channel retainers being held in a spaced apart relationship by at least one resilient member comprising an arch, the pad being attached or molded to the resilient member, and the tongue retainer being attached to two of the channel retainers by two secondary resilient members.

According to a still further embodiment of the invention, there is provided a retracting device for retracting a user's lips having at least one formation for accommodating a dental treatment composition, for example, a whitening composition. In one aspect, the formation may include at least one u-shaped channel may be configured to accommodate the lower, the upper or both sets of a user's teeth. The u-shaped channel supports the channel retainers in substantially fixed spatial relation with respect to one another. In another aspect, the arch of the retracting device may be configured to accommodate a u-shaped channel.

According to still another embodiment of the invention, a retracting device having at least one formation such as a wing-like member may be held in place by the natural compression of the lips of a subject. The wing-like members provides positioning and alignment to at least one mating formation on an imaging apparatus. The configuration enables patients to hold a position during imaging with comparatively little effort.

In one aspect, a retracting device includes at least one formation such as a passively held portion to anchor it to a subject of dental imaging. The device further includes a first alignment formation coupled to the passively held portion where the first alignment formation provides alignment to at least one dental feature; and the device may also include a second alignment formation coupled to an imaging device where the second alignment formation is shaped and configured to mate with at least one formation in the imaging device and both the first and second alignment formations serve to align the imaging device in a substantially fixed position with respect to the at least one dental feature. The device may include a film holder coupled to the passively held portion. The film holder is adapted to hold an imaging film, or an imaging sensor, for imaging at least one dental feature.

In one embodiment, the retracting device may be a single-use device, and the imaging film, or imaging sensor may be integrally formed with the holder.

In one aspect, a retracting device of the invention may be fitted with a formation such as a tab for grasping and for facilitating insertion and removal of the device.

Other alternatives and embodiments for practicing the invention are also described herein and further discussed below in the Detailed Description section.

### BRIEF DESCRIPTION OF THE DRAWINGS

FIGS. 1 and 1b depict a semi-schematic perspective view of a lip retracting device provided in accordance to one embodiment of the present invention;

FIG. 1a depicts a semi-schematic perspective view of an alternative lip retracting device provided in accordance to another embodiment of the present invention;

FIG. 2 depicts a semi-schematic bottom plan view of the lip retracting device of FIG. 1a fitted into a device, such as an output port, a light guide, an imaging device, a light source or an examination cone;

FIG. 3 depicts a semi-schematic bottom plan view of the lip retracting device FIG. 1:

FIG. 4 depicts a semi-schematic side view of the lip retracting device of FIG. 1 taken along line A-A of FIG. 3;

FIG. 5 depicts a semi-schematic top plan view of the lip retracting device of FIG. 1;

FIG. 6 depicts a semi-schematic side view of the lip retracting device of FIG. 4 taken along line B-B of FIG. 4;

FIG. 7 depicts a semi-schematic side view of the lip retracting device of FIG. 5 taken along line C-C of FIG. 5;

FIG. 8 depicts a semi-schematic perspective view of an alternative lip retracting device provided in accordance to another embodiment of the present invention;

FIG. 9 depicts a semi-schematic front view of the lip retracting device of FIG. 1 worn by a user/patient;

FIG. 10 depicts a semi-schematic front view of the lip retracting device of FIG. 8 worn by a user/subject;

FIG. 10a depicts another embodiment of a retracting device of FIG. 8 in service on a user/subject;

FIG. 11 depicts a semi-schematic top plan view of the lip retracting device of FIG. 1a;

FIG. 11a depicts a more detailed semi-schematic top view of the lip retracting device of FIG. 11;

FIG. 11b shows a semi-schematic bottom plan view of the lip retracting device of FIG. 11a fitted into a light guide;

FIG. 11c shows an embodiment of a light guide according to an embodiment of the present invention;

FIG. 12 depicts a semi-schematic side view of the lip retracting device of FIG. 11;

FIG. 13 depicts a semi-schematic front view of a light guide with slots;

FIG. 14 depicts a semi-schematic side view of the lip retracting device of FIG. 11 fitted with pads;

FIG. 15 depicts a semi-schematic top view of a pad having a clam-shell configuration;

FIG. 16 depicts a perspective view of an embodiment of a lip retracting device including a u-shape channel;

FIG. 16a depicts the rear view of the embodiment of FIG. 16;

FIG. 17 depicts a perspective view of an embodiment of a lip retracting device including a u-shape channel;

FIG. 18 depicts a perspective view of an embodiment of a lip retracting device having a u-shape channel with a tab;

FIG. 19 shows, in perspective view, a lip retracting device accommodating both lower and upper sets of teeth according to one embodiment of the invention;

FIG. 20 shows an exploded view of the combination of a lip retracting device with the light guide and a lamp;

FIG. 21 shows, in perspective view, a dental illumination system suitable for use with embodiments of the invention;

FIG. 22 shows, in perspective view, a lamp head of a dental illumination system;

FIG. 23 shows a top view of the lip retracting device of FIG. 16 mated with a light guide according to principles of the invention;

FIG. 24 shows, in perspective view, a dental illumination frame of a dental illumination system of the present invention;

FIG. 25 shows, in exploded perspective view, a light guide including a flexible cushion and an illumination frame according to one embodiment of the invention;

FIG. 25a shows an embodiment of an illumination frame including a heat sink;

FIG. 26 shows, in perspective view, an embodiment of an illumination frame of the present invention;

FIG. 27 shows, in perspective view, a dental whitening or curing lamp according to one embodiment of the invention;

FIG. 28 shows a top view of an illumination frame mated with a lip retracting device according to one embodiment of the invention;

FIG. 28a shows an illumination frame having formations according to an embodiment of the present invention;

FIG. 28b shows another embodiment of an illumination frame mated with a lip retracting device according to one embodiment of the invention;

FIG. 28c depicts a semi-schematic front view of an exemplary lip retracting device coupled to a patient/user according to one embodiment of the invention;

FIG. 29 shows, in perspective view, a retracting device with extended wings according to an embodiment of the invention;

FIG. 29a shows, in perspective view, a retracting device including targets according to one embodiment of the invention;

FIG. 29b shows, in perspective view, a retracting device with extended wings and targets according to an embodiment of the invention;

FIG. 29c shows, in perspective view, a retracting device including a film holder according to an embodiment of the invention;

FIG. 29d shows, in perspective view, an alternative configuration of a retracting device;

FIG. 29e shows in perspective view, another embodiment of a retracting device;

FIG. 30 shows, in perspective view a stationary imaging stand according to one embodiment of the invention;

FIG. 30a shows an embodiment of the invention including a dental support structure and a dental imaging fixturing system; and

FIG. 30b shows another embodiment of the invention including a dental support structure and a dental imaging fixturing system.

### DETAILED DESCRIPTION

The detailed description set forth below in connection with the appended drawings is intended as a description of the presently exemplified embodiments of a retracting device provided in accordance with the present invention and is not intended to represent the only forms in which the present invention may be constructed or utilized. The description sets forth the features and the steps for constructing and using the retracting device of the present invention in connection with the illustrated embodiments. It is to be understood, however, that the same or equivalent functions and structures may be accomplished by different embodiments that are also intended to be encompassed within the spirit and scope of the invention. Also, as denoted elsewhere herein, like element numbers are intended to indicate like or similar elements or features.

The retracting device includes inter-engaging and/or non inter-engaging formations. Inter-engaging formations include those formations that engage a device, an apparatus or tool with at least one corresponding formation in another device, apparatus or tool. Non-inter-engaging formations include those formations that bring such device, apparatus or tool into close proximity with at least a portion of a subject's mouth.

The word formation as used herein in relation to a dental system such as a light system, a light guide, an imaging system, a dental treatment composition, an imaging system an apparatus adapted for aspiration, a retracting device, a spacer, a support system, or dental tools adapted for aspiration, such as aspirators or suction tubes, refers to the portion of the dental system which is adapted to inter-fit with a corresponding portion of an adjoining dental system, component or a subject's mouth. A formation thus includes at least a portion of any of the above listed articles and may be formed or shaped by molding, or the formation may be formed separately and then subsequently assembled with the respective articles.

Suitable inter-engaging formations include tongues and grooves, posts and sockets, swingable hooks and sockets, resilient clips and sockets, clips and protrusions or depressions, tongues or wing-like members and slots, ball and cavity, ball and socket, some of which are more specifically exemplified in detail below. Non-inter-engaging formations include dental trays, imaging film holders, and other features adapted to position any dental treatment or imaging material in a patient's mouth.

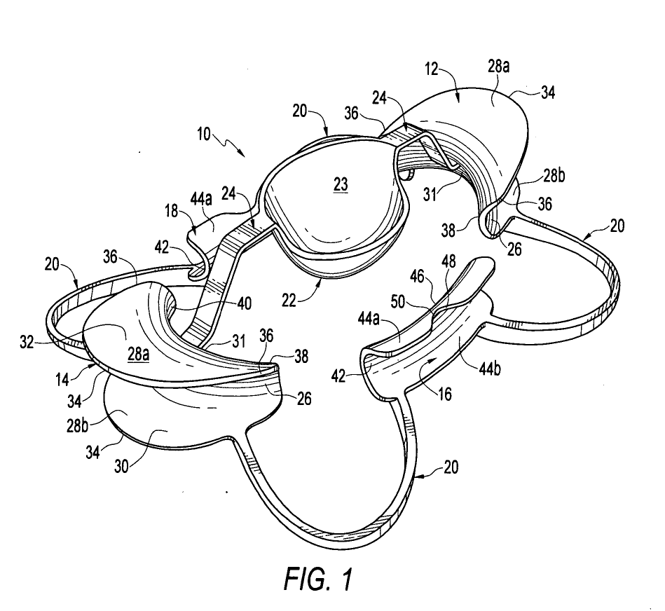

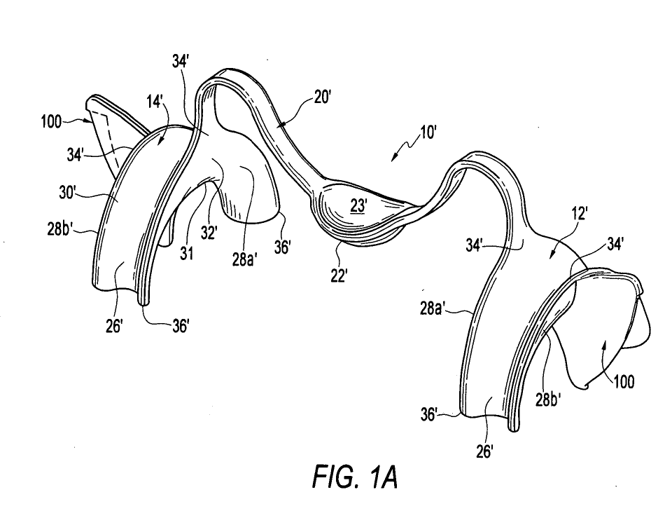

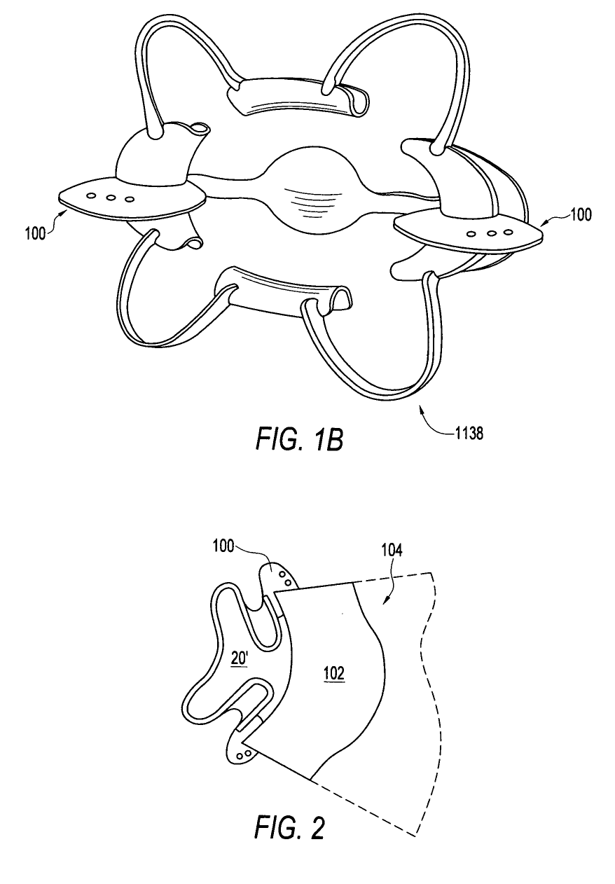

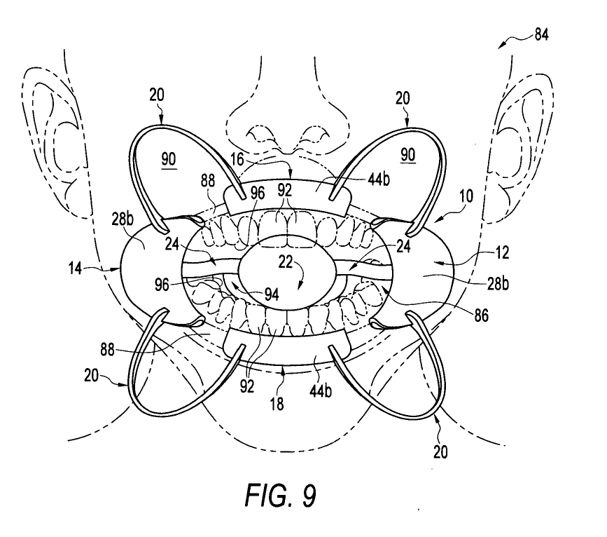

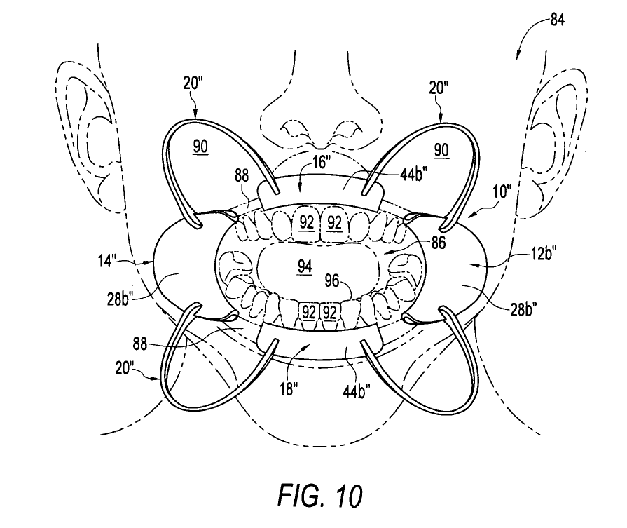

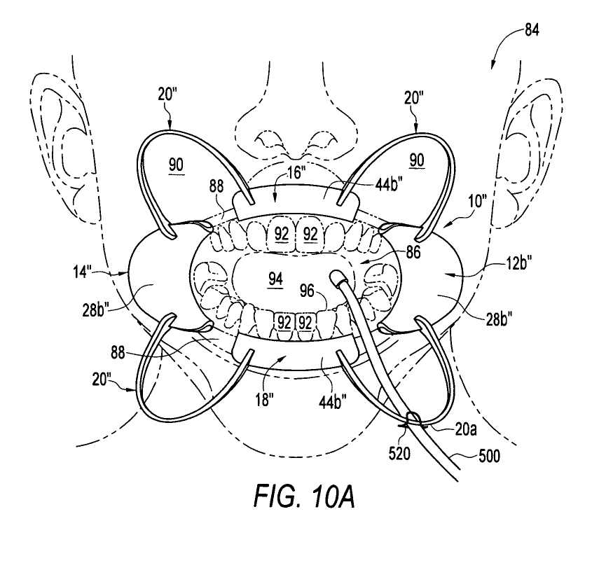

Referring to FIG. 1, a lip retracting device for retracting the upper and lower lips (herein “lips”) for facilitating examination and/or treatment of the mouth and/or teeth provided in accordance to one practice of the present invention is generally shown and designated 10. The lip retracting device 10 may also be known as a tongur cup, and includes four spaced apart channel retainers 12, 14, 16, 18, also known as flanges, for retaining four corresponding portions of the lips for examination and/or treatment of the mouth or teeth. When used, the lip retracting device 10 draws back the lips, which retracts the cheeks, to expose the mouth so that a health care professional can more easily see the teeth and work on the teeth and/or mouth, such as exemplified in FIGS. 9 and 10.

The four channel retainers include two side channel retainers 12, 14 for retaining the ends of the lips, approximately where the upper and the lower lips intersect, and two lip channel retainers 16, 18 for retaining the mid-section of the upper and lower lips. More particularly, the four channel retainers or flanges 12, 14, 16, 18 are adapted to cup the lips and bias them open to expose the teeth for treatment and/or examination, such as exemplified in FIGS. 9 and 10, as noted above.

A plurality of resilient members 20 are incorporated in the lip retracting device 10 to interconnect the four channel retainers 12, 14, 16, 18 together and to also function as biasing means. In the ready position (before insertion of the lip retracting device into the mouth), the resilient members 20 are arched outwardly with respect to the center portion of the retracting device 10. As further discussed below, when the retracting device 10 is inserted into the mouth and the four channel retainers 12, 14, 16, 18 cup respective portions of the lips, the resilient members 20 provide a retractive force to retract the lips radially outwardly for examination and/or treatment.

An optional tongue retainer 22 is shown approximately centrally positioned relative to the four channel retainers 12, 14, 16, 18. The tongue retainer may also be positioned asymmetrically about the two channel retainers 16 and 18. The tongue retainer 22 includes a trough 23 and is attached to two channel retainers 12, 14 by a pair of secondary resilient members 24. When incorporated, the tongue retainer 22 and the secondary resilient members 24 cooperate to block the tongue and limit the tongue to the back vicinity of the mouth, thus enabling access to the lingual portion or the back portion of the teeth for examination and/or treatment. In short, the tongue retainer is configured to minimize interference by the tongue during treatment and/or examination by a health care professional.

Any of the resilient members 20 may be formed as a single piece, integrally molded or attached by an adhesive or heat sealing to, for example, the outside side surface 28b and 44b of a pair of adjacent channel retainers 14, 16 or it may be formed in two halves separately and connected to the mid-portion, also integrally molded or attached by an adhesive or heat sealing to the outside side surface 28b and 44b of the adjacent channel retainers 14 and 16.

As shown, the side channel retainers 12, 14 resemble a curvilinear c-channel in that they include an arcuate race 26 and two channel side walls 28a, 28b. The channel side walls 28a, 28b resemble a bell shape and include a maximum wall dimension at approximately the mid-point 34 and two smaller tapered tips 36 at the ends thereof. In one embodiment, the inside side wall 28a, which is intraoral as further discussed below, is slightly larger relative to the outside side wall 28b. However, the relative dimensions can be reversed or can be the same without deviating from the functionality of the retracting device 10.

The side channel retainers 12, 14 further include an interior surface 30 and an exterior surface 32. The arcuate race 26 includes a curved portion 31 adapted to mimic the curvature of the side of the lips when the lips are in the opened position. Because this curvature may vary depending on the size and age of the user or patient, the retracting device 10 may be implemented with varying radii of curvatures of portion 31 to fit the varied shape of the particular user/patient. The arcuate race 26 may also include an irregular curvature or two or more different radii of curvatures. For example, the lower region 38 of the curve portion 31 may have a larger radius than the upper region 40 or vice versa. If implemented, the irregular curvature may vary the amount of retraction of the portion of the lip that is seated within the arcuate race to vary the amount of retraction between those portions of the lip. The two lip channel retainers 16, 18 may also have different radii of curvatures, similar to the side channel retainers 12, 14.

As shown, the lip channel retainers 16, 18, like the side channel retainers 12, 14, resemble a curvilinear c-channel in that they include an arcuate race 42 and two channel side walls 44a, 44b. In one embodiment, the radius of curvature of portion 46 of the lip channel retainers is larger than the radius of curvature of portion 31 of the side channel retainers 12, 14. The larger radius of of portion 46 enables the lip channel retainers 16, 18 to conform to the contour of the upper and lower lips near the frenum, which is more planar relative to the side of the lips. Depending on the size and age of the intended user/patient, the radius of curvature of portion 46 of the lip channel retainer 16, 18 may also vary.

As shown, a frenum release 48 is incorporated in the inside side walls 44a of the lip channel retainers 16, 18 for providing relief to the frenum of the upper and lower lips. In one embodiment, the frenum release 48 may include a partial oval shaped cutout having a size sufficient to provide clearance for the frenum. In other words, the frenum release 48 may be such that the lowest most portion 50 of the frenum release only slightly touches the frenum when in use, for example, or for example, does not touch the frenum. Although the oval shaped cutout is shown for the frenum release 48, a partial circle, a rectangular cutout, a square cutout, or other geometrical shaped cutout may also be incorporated without deviating from the function of the frenum release.

A lip retracting device, such as presently shown as 10, may be made by injection molding or casting a thermoplastic material such as polypropylene, polyethylene, polystyrene, polyester, polycarbonate or the like. It may also be made out of biocompostable or biodegradable polymers including polyesters such as a polylactic acid resin (having L-lactic acid and D-lactic acid), and polyglycolic acid(PGA); polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers; and polyester/urethane resin. More for example, the lip retracting device may be made by injection molding polypropylene and may be a smooth and transparent finish. In another embodiment, the device may be opague and colored, including white color.

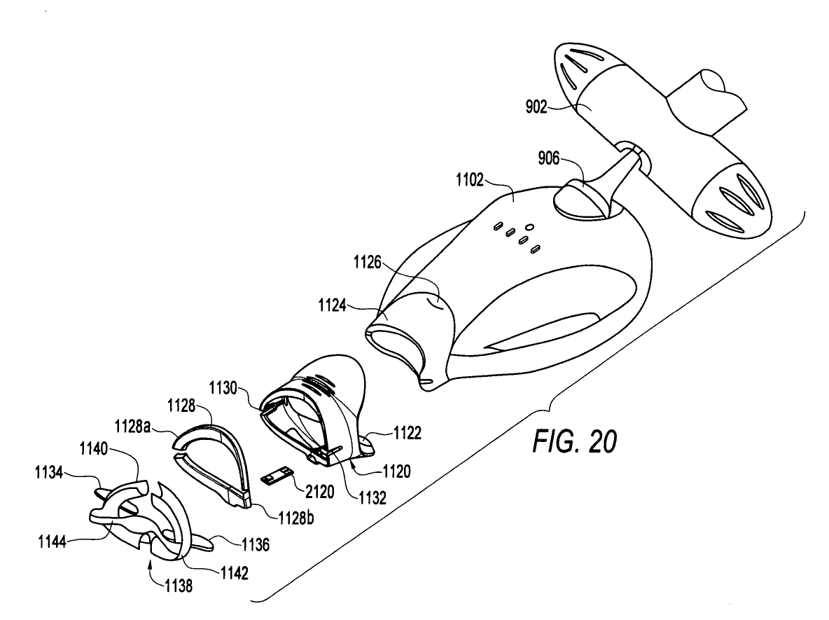

As shown in FIG. 1b, two formations, for example, two wing-like flanges 100 may be incorporated in the lip retracting device of FIG. 1. These wing-like flanges 100 may be molded or cast integrally with, or attached by an adhesive or heat sealing to, the side channel flanges or retainers 12, 14, and may be constructed of the same or different material as the channel flanges or retainers, or other parts of the retracting device, including the materials mentioned above, or of a more sturdy polymeric material or composite. Additionally, it may also be opaque or colored even if the rest of the lip retracting device may be colorless or clear. The wing-like flanges 100 may be designed for fitting into a pair of formations, such as slots 1130, 1132 formed in the output port, an imaging device, a lamp system, or a light guide 1120 of a lamp system 1102 used in a whitening process or to the slots in any examining device, such as that shown in an exemplary illumination system of FIG. 20, an exploded view of a combination of a lip retracting device 1138, a light guide 1120 and a lamp system 1102. Another exemplary illumination or lamp system and the use of which is disclosed in Ser. No. 10/715,681, filed Nov. 17, 2003, which is expressly incorporated herein by reference as if set forth in full.

In another embodiment, instead of a light guide 1120, as shown in FIG. 20, an examining device such as a cone-like structure mentioned above, may be configured to fit over the outlet of the lamp 104 and the wing-like flanges 100 on the lip retracting device 1138, such as shown in FIG. 13. The wing-like flanges 100 may be configured to interact with formations, such as slots 112 on the cone, to thereby provide a consistent and controlled gap between the lamp 104 and the teeth of the patient to be treated or examined.

Referring now to FIG. 1a, there is shown a lip retracting device 10′ for retracting the lips for facilitating examination, imaging of the mouth and/or teeth, and/or the whitening or curing process provided in accordance to one practice of the present invention. The retractor includes two spaced apart channel retainers or flanges 12′, 14′, for retaining two corresponding portions of the lips for examination and/or treatment of the mouth and/or teeth. When used, the lip retracting device 10′ similarly draws back the lips, which retracts the cheeks, to expose the mouth so that a health care professional can more easily see the teeth and work on the teeth and/or mouth.

Like the retracting device of FIG. 1 and 1a, the two channel retainers 12′, 14′ may be adapted for retaining the ends of the lips, approximately where the upper and the lower lips intersect. More particularly, the two channel retainers or flanges 12′, 14′ are adapted to cup the lips and bias them open to expose the teeth for treatment and/or examination.

A resilient member 20′ is incorporated in the lip retracting device 10′ to interconnect the two channel retainers 12′, 14′ together and to function as biasing means. The resilient member 20′ has, for example, two arches, as shown, one on either side of the center portion 22′. The resilient member 20′ may be formed as a single piece, integrally molded or attached by an adhesive or heat sealing to the inside side walls 28a′ of the channel retainers 12′, 14′, or it may be formed in two halves separately and connected to the mid-portion 22′, also integrally molded or attached by an adhesive or heat sealing to the inside side wall 28a′ of the channel retainers 12′, 14′. In the ready position (before insertion of the lip retracting device into the mouth), the resilient members 20′ may be arched outwardly with respect to the center portion of the lip retracting device 10′. As further discussed below, when the lip retracting device 10′ is inserted into the mouth and the two channel retainers 12′, 14′ cup respective portions of the lips, the resilient members 20′ provide a retractive force to radially retract the lips outward for examination and/or treatment. This lip retracting device is especially useful for the whitening process.

An optional tongue retainer 22′ may also be approximately centrally positioned relative to the two channel retainers 12′, 14′. Like the tongue retainer 22 of FIG. 1, the tongue retainer 22′ of the present embodiment may also include a trough 23′. Further, it may be integrally formed on the mid-portion of the resilient member 20′ and thus may be attached to the channel retainers 12′, 14′ via resilient member 20′. When incorporated, the tongue retainer also blocks the tongue and limits the tongue to the back vicinity of the mouth, thus enabling access to the lingual portion or back of the teeth for examination and/or treatment, just like that discussed above, to minimize interference by the tongue during treatment and/or examination by a health care professional.

In this embodiment, the resilient member 20′ acts not only to connect the channel retainers and to bias them, but also to connect the tongue retainer to the channel retainers. If the tongue retainer 22′ is not incorporated, the resilient member 20′ would simply extend from one channel retainer 12′ to another channel retainer 14′ at a substantially uniform width.

The channel retainers 12′, 14′ also resemble a curvilinear c-channel in that they include an arcuate race 26′ and two channel side walls 28a′, 28b′. The channel side walls 28a′, 28b′ also resemble a bell shape and include a maximum wall dimension at approximately the mid-point 34′ and two smaller tapered tips 36′ at the ends thereof. In one embodiment, the inside side wall 28a′, which is also intraoral, may be slightly larger relative to the outside side wall 28b′. However, the relative dimensions may again be reversed or may be the same without deviating from the functionality of the lip retracting device 10′.

Similar to FIGS. 1 and 1b, the side channel retainers 12′, 14′ further include an interior surface 30′ and an exterior surface 32′. The arcuate race 26′ includes a radius of curvature of portion 31′ adapted to mimic the curvature of the side of the lips when the lips are in the opened position. Also, because this curvature may again vary depending on the size and age of the user or patient, as noted above, the lip retracting device 10′ may be implemented with varying radii of curvatures of portion 31′ to fit the varied shape of the particular user/patient. The arcuate race 26′ may also include an irregular curvature or two or more different radii of curvatures. For example, the lower region 38′ of the radius of curvature of portion 31′ may have a larger radius than the upper region 40′ or vice versa. If implemented, the irregular curvature can vary the amount of retraction of the portion of the lip that is seated within the arcuate race to vary the amount of retraction between those portions of the lip.

Similarly, the lip retracting device 10′ may also be made by injection molding or casting a thermoplastic material such as those already mentioned. For example, the lip retracting device 10′ may be made by injection molding pigmented polypropylene and may be opaque white or colored having a smooth finish, or it may be made by injection molding clear polypropylene, and has a smooth finish.

Additionally, FIG. 1a also shows two formations such as wing-like flanges 100, extending from the outside side wall 28b′ of the channel retainers 12′, 14′. The wing-like flanges 100 may also be molded or cast integrally with the channel flanges or retainers 12′, 14′, or attached, as discussed above As further discussed above and in more detailed below, the wing-like flanges may be designed for fitting the lip retracting device 10′ to the formations, such as slots, formed on a cone section of an output port or a light guide of a lamp source used in a teeth whitening, curing, or imaging process, or to the slots in any examining device. As an example, FIG. 2 shows a bottom view of the lip retracting device 10′ with its wing-like flanges 100 engaging the slots on, for example, a light guide 102, which is attached to a lamp 104 shown in dash-dot lines.

Similarly, as mentioned above, the wing-like members or flanges 100 may be made of the same or different material as the rest of the lip retracting device, or the channel flanges or retainers, including the materials mentioned above, or of a more sturdy polymeric material or composite. Additionally, it may also be opaque or colored even if the rest of the lip retracting device may be colorless or clear.

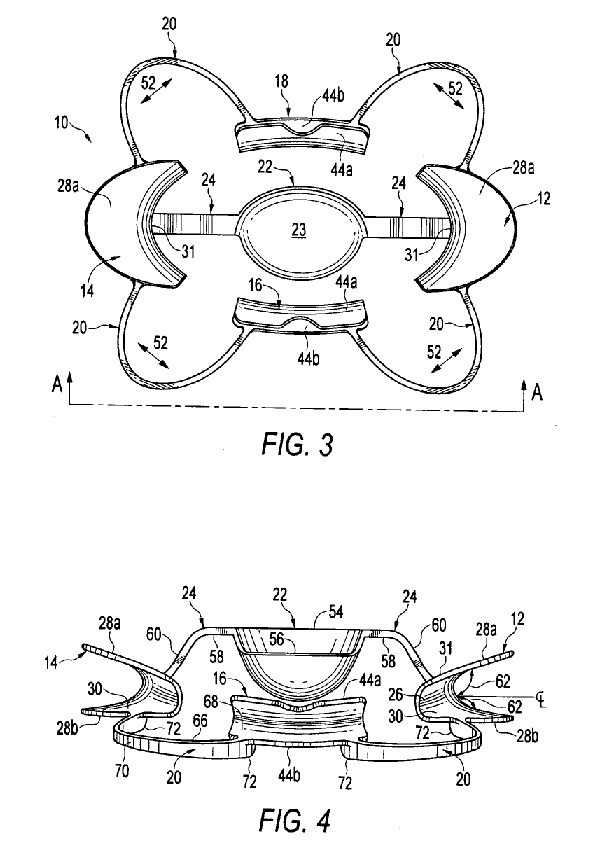

FIG. 3 is a semi-schematic bottom plan view of the lip retracting device 10 of FIG. 1. The lip retracting device 10 is shown in a ready-to-use configuration, a configuration in which the four resilient members 20 bias the four channel retainers 12, 14, 16, 18 outwardly 52 away or in a spaced relationship from one another. Similarly, the two secondary resilient members 24 bias the tongue retainer 22 away from the plane defining the position of the four channel retainers 12, 14, 16, 18 (approximately perpendicularly towards the viewer). Hence, as further discussed below, when the lip retracting device 10 is placed in the mouth during service, the four channel retainers 12, 14, 16, 18 are adapted to cup the lips and the four resilient members 20 are adapted to spread the lips open due to the resiliency of the resilient members 20 to expose the labial or front portions of the teeth, as shown in FIGS. 9 and 10. Similarly, the tongue retainer 22 is adapted to block the tongue and the two secondary resilient members 24 are adapted to limit the tongue to the back region of the mouth, towards the throat, to further expose the lingual or back portions of the upper and lower teeth, as shown in FIG. 9.

When in service inside a patient's mouth, the inside side walls 28a, 44a, the secondary resilient members 24, and the tongue lip retracting device 22, including the trough 23, are configured to be intraoral while the outside side walls 28b, 44b, as seen in FIG. 4, and the four resilient members 20 are configured to be extraoral. As is readily apparent, the four resilient members 20 are integrally molded to the outside side walls 28b, 44b to not interfere with the insertion of the lip retracting device into the mouth.

FIG. 4 is a semi-schematic side view of the lip retracting device of FIG. 3 taken at line A-A. FIG. 4 shows the tongue retainer 22 including an upper rim 54 and a lower rim 56. The upper rim 54 is positioned higher relative to the lower rim 56 (i.e., protruded further into the mouth than the lower rim when the lip retracting device is in service) and is integrally molded or attached to the two secondary resilient members 24. Alternatively, the tongue retainer 22 may have two even rims to retain the tongue evenly along the upper and lower surfaces of the tongue.

The two secondary resilient members 24 are shown having a generally horizontal section 58 extending from the upper rim 54 and a sloped section 60 connected to the horizontal section 58 and to the curve portion 31 of the two side channel retainers 12, 14. Alternatively, the two secondary resilient members 24 may include a single sloped section that connects to both the upper rim and the curve portion.

Referring specifically to the right side channel retainer 12, there is shown a channel centerline ℄, which divides the channel retainer at approximately the mid-point of the race 26. From the perspective of the centerline ℄, it can be observed that the inside side wall 28a is angularly spaced a greater distance 62 than the angular position 64 of the outside side wall 28b. The surface area of the outside side wall 28b is also smaller relative to the inside side wall 28a. Among other things, this offset or non-symmetrical configuration is believed to conform better to the physical characteristics of the cheeks and the lips, which translate to a more comfortable fit when the lip retracting device is in service. In other words, the channel retainers 12, 14 shown are not half-circles or symmetrical about a line or a point to accommodate the non-symmetrical features of the lips. However, it is possible to make the side channel retainers 12, 14 symmetrical and/or oversized and still provides a comfortable fit.

Also shown in FIG. 4 is the manner in which the resilient members 20 are integrally molded or attached to the outside side surface 28b of the side channel retainers 12, 14 and the outside side surface 44b of the lip channel retainer 16. In one particular embodiment, the resilient members 20 each has an upper edge 66 that is flushed with, flat with, or otherwise smoothly transitioned to the interior surface 30 of the side channel retainers 12, 14 and the interior surface 68 of the lip channel retainer 16. This arrangement allows the lip retracting device 10 to be worn without sharp edges projecting or protruding against the inside surface of the lips and the cheeks. However, a slight deviation in the transition between the upper edge 66 and the interior surfaces 30, 68 of the channel retainers 12, 14, 16 may still be acceptable as the lips and the cheeks are pliable and can accommodate any minor deviation without being overly irritating to the user/patient.

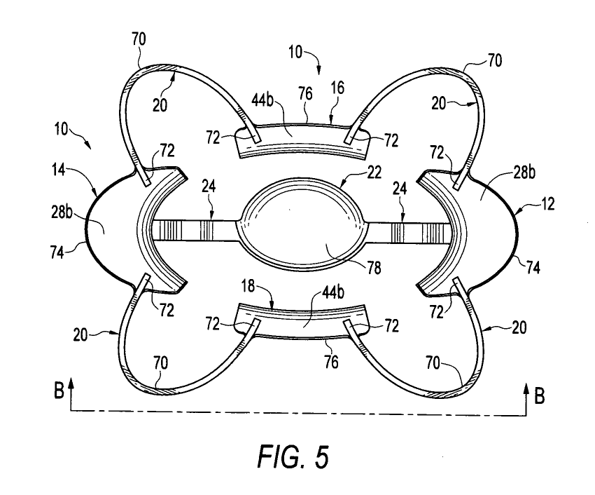

Referring now to FIG. 5, there is shown an exemplary semi-schematic top plan view of the lip retracting device of FIG. 1. As shown, the end points 72 of the resilient members 20 extend past the edges 74 of the side channel retainers 12, 14 and the edges 76 of the lip channel retainers 16, 18 for bonding or molding integrity between the resilient members 20 and the channel retainers 12, 14, 16, 18. However, the amount of overlap between the end points 72 and the edges may vary depending on the particular materials used for molding the lip retracting device 10, which may have sufficient strength without substantial or any overlapping. The tongue retainer 22 is shown as having an oval shaped contour and a smooth exterior surface 78. However, a circular, square, rectangular, or other geometrical shape tongue retainer 22 may also be used without deviating from the scope of the present invention.

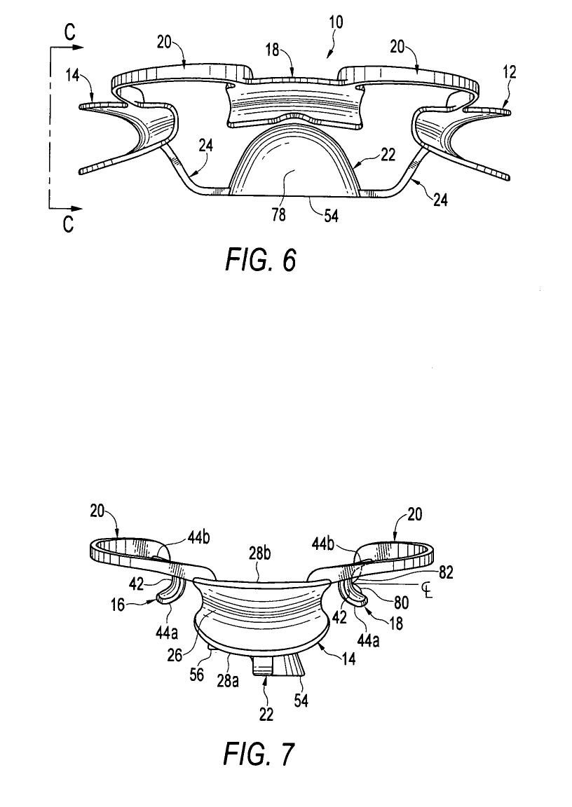

FIG. 6 is a semi-schematic side view of the lip retracting device of FIG. 5 taken at line B-B. Assuming that the side channel retainers 12, 14, and the lip channel retainers 16, 18 define a curved plane, as shown; the upper rim 54 of the tongue retainer 22 is subjacent to the curved plane. As previously discussed, the subjacent configuration and the depth of the trough 23 (FIG. 3) enable the tongue retainer 22 to limit and retain the tongue in the back of the mouth to further expose the lingual surface of the teeth.

FIG. 7 is a semi-schematic side view of the lip retracting device 10 of FIG. 6 taken at line C-C. Similar to the side channel retainer 12 of FIG. 4, the lip channel retainer 18 includes a lip channel centerline ℄ or mid-point, which divides the lip channel retainer at approximately the centerline of the arcuate race 42. From the perspective of the centerline ℄, it can be observed that the inside side wall 44a is angularly spaced a smaller distance 80 than the angular position 82 of the outside side wall 44b relative to the centerline. Among other things, this offset or non-symmetrical configuration is adapted to conform to the physical nature of the lips, which translate to a more comfortable fit when the lip retracting device is in service.

Alternatively, the particular relationship may switch such that the angular positions of the inside and outside walls 44a, 44b relative to the centerline are the same or reversed, as discussed above for the side channel retainers 12, 14.

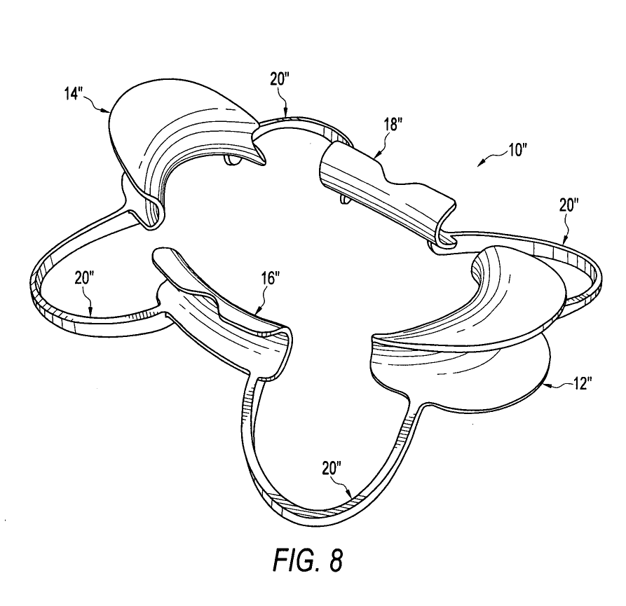

FIG. 8 is a semi-schematic perspective view of an alternative lip retracting device 10″ provided in accordance to another embodiment of the present invention. As shown, the lip retracting device includes two side channel retainers 12′, 14′, two lip channel retainers 16′, 18′, and four resilient members 20′. In one embodiment, the alternative lip retracting device 10′ is identical to the lip retracting device 10 shown in FIGS. 1, 1b, 3-7 except that the alternative lip retracting device 10′ does not include a tongue retainer. Accordingly, the description set forth above for the lip retracting device 10 applies for the alternative lip retracting device 10′ less the tongue retainer.

Similarly, although not specifically shown, lip retracting device 10′, as seen in FIG. 1a, may also be made without the optional tongue retainer.

FIG. 9 is an exemplary semi-schematic top plan view of the lip retracting device 10 of FIG. 1 in use on a patient or user 84. As shown, the lip retracting device 10 engages the user's mouth 86 to retract the user's lips 88 and cheeks 90. Once in position, the user's mouth 86, and particularly the teeth 92, is exposed for examination and/or treatment by a health care professional. More specifically, the side channel retainers 12, 14 engage the side of the mouth, the lip channel retainers 16, 18 engage the upper and lower lips 88, and the resilient members 20 bias the four channel retainers, which bias the lips 88 and cheeks 90 open, to expose the teeth and the inside of the mouth 86. In the lip retracting device used position, the outside side surfaces 28b, 44b and the resilient members 20 are exposed extraorally of the mouth.

The tongue retainer 22 is shown engaged to the tongue 94 and relegates the tongue to the back region of the mouth 86. When incorporated, the tongue retainer 22 is configured to further expose the lingual surface 96 of the teeth for examination and/or treatment.

The lip retracting device 10 may be installed on the lips 88 by first placing the upper lip over the inside side wall 44a and into the race 42 of the upper lip channel retainer 16. The two side channel retainers 12, 14 are then squeezed together and placed into the mouth, either concurrently or one at the time, until the sides of the lips fit over the inside side walls 28a of the side channel retainers 12, 14 and into the race 26. Finally, the lower lip channel retainer 18 is squeezed and placed over the lower lip 88 with the lower lip engaging the race 42 of the lip channel retainer 18. Once installed, the tongue retainer 22, if present, automatically aligns with the tongue 94 to block the tongue from maneuverability. The lip retracting device 10 may also be installed by reversing the steps discussed above or squeezing all four channel retainers at the same time and fitting the lips over the channel retainers.

FIG. 10 is an exemplary semi-schematic top plan view of the alternative lip retracting device 10′ of FIG. 8 in use on a patient or user 84. Like the lip retracting device 10 in FIG. 9, the alternative lip retracting device 10′ engages the lips to retract the lips 88 and the cheeks 90 to thereby expose the teeth 92 for examination and/or treatment. However, unlike the lip retracting device 10 of FIG. 9, the alternative lip retracting device 10′ does not incorporate a tongue retainer. Thus, the tongue 94 is shown free to move within the oral cavity of the mouth 86.

In some embodiments, the retracting devices may include at least one formation adapted for mating with corresponding formations on an apparatus or tool adapted for aspiration, such as a suction tube and/or air/gas aspirator, or others, for positioning such apparatus in proximity to at least a portion of or inside a subject's mouth. One exemplary embodiment is shown in FIG. 10a, which shows another embodiment of FIG. 10, where the formation is shown as a protrusion 20a, such as a post or a bump, disposed on one of the resilient members 20, adapted for mating with a clip 520 disposed on a suction tube 500.

The formation may also be a depression, such as a dent, adapted for accepting a clip.

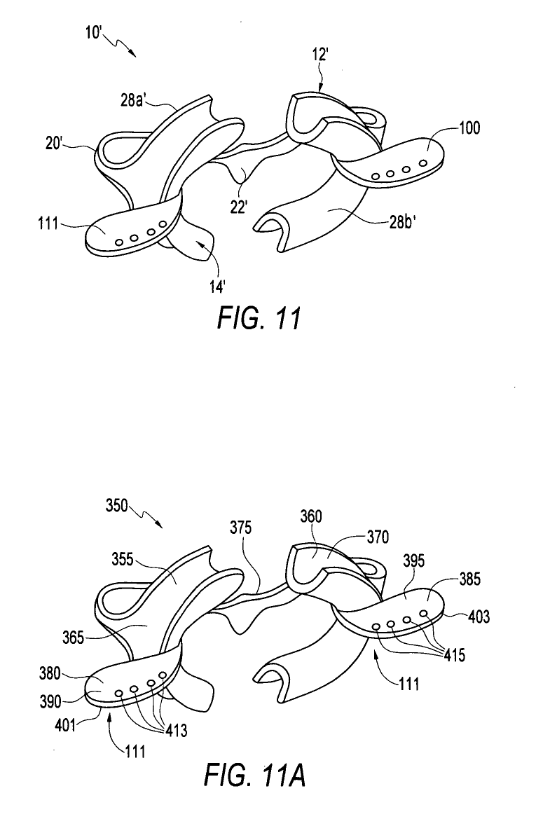

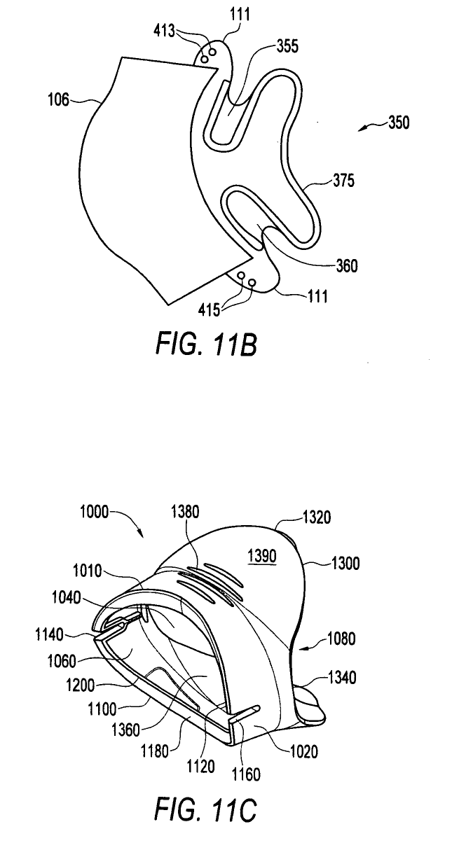

FIG. 11 is a semi-schematic bottom plan view of the lip retracting device 10′ of FIG. 1a. The lip retracting device 10′ is in a ready position, which is the position in which the arches of the resilient member 20′ bias the two channel retainers 12′, 14′ towards each other to form a substantially circular shape for an unobstructed view of the inside of the mouth. At the same time, the resilient member 20 , bias the tongue retainer 22′ away from the plane defining the position of the two channel retainers 12′, 14′ (approximately perpendicularly towards the viewer). Hence, as further discussed below, when the lip retracting device 10′ is placed in the mouth during service, the two channel retainers 12′, 14′ are adapted to cup the lips and the arches formed by the resilient member 20′ are adapted to spread the lips open due to the resiliency of the resilient members 20′ to expose the labial or front portions of the teeth. Similarly, the tongue retainer 22′ is adapted to block the tongue and the resilient members 20′ are adapted to limit the tongue to the back region of the mouth, towards the throat, to further expose the lingual or back portions of the upper and lower teeth. The wing-like members 100 are positioned outside of the mouth for fitting the lip retracting device 10′ to the slots formed in any output port or light guide of a lamp source, an imaging device or an examination device such as a cone, as shown generically in FIGS. 2 and 13, as mentioned above. Note that the tongue retainer 22′ is shown as a generally flat and rectangular plate incorporated at the mid-point of the resilient member 20′, which is an alternative to the tongue retainer 22′ shown in FIG. 1a in which a trough 23′ is incorporated.

The lip retracting device 10′ is configured to fit into the mouth in the orientation shown in FIG. 11. In other words, the inside side walls 28a′, and the tongue retractor 22′ are configured to be intraoral while the outside side walls 28b′, the resilient member 20′ and the wing-like members 100 are configured to be extraoral.

FIG. 11a shows a lip retracting device 350 includes first 355 and second 360 semicircular “U”-shaped channels adapted to receive the lips of a dental patient adjacent to respected internal surfaces 365, 370 thereof. This is similar to FIG. 1a, but in a ready-to-use configuration. In this ready-to-use configuration, some of the structures takes on a different form than that shon in a not-ready-to-use mode. A support member 375 may be mutually coupled to the “U”-shaped channels 355, 360 and adapted to support the “U”-shaped channels 355, 360 in substantially fixed spatial relation with respect to one another. According to one embodiment of the invention as shown in FIG. 11a, the support member 375 also supports a tongue-cap adapted to retain a patient's tongue and shield the same from incidental illumination.

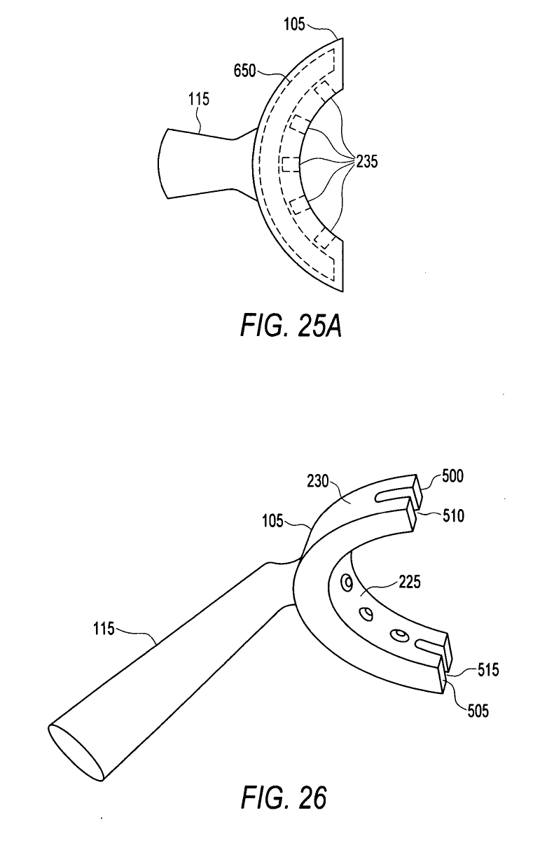

According to one embodiment of the invention, a pair of interface wings 111 is coupled to the “U”-shaped channels 355, 360 respectively. According to this embodiment, the interface wings 111 each include a respective upper surface 390, 395 and a respective lower surface 401, 403. According to one embodiment of the invention, upper surface 390 is disposed substantially parallel in relation to lower surface 401 and upper surface 395 is disposed substantially parallel in relation to lower surface 403. Pursuant to one embodiment of the invention, the interface wings 111 have a first plurality of ticks or holes 413 and a second plurality of ticks or holes 415 respectively. The ticks or holes are adapted to facilitate maintaining a particular alignment of, for example, an illumination frame (as shown in FIG. 26) 105, or imaging devices with respect to the lip retracting device 350.

According to one embodiment of the invention, interface wings 111 are adapted to be received within slots 236′, 238′ of a light guide 106, as shown in, for example, FIG. 11b, respectively. By pressing the lip retracting device 350 toward the front edge of light guide 106, the interface wings 111 are urged into slots 236′ and 238′, whereby the orientation and position of the lip retracting device 350 with respect to the light guide 106 is substantially fixed. Consequently, to the extent that a patient's lips effectively serve to couple the head and teeth of the patient in fixed relation to the lip retracting device 350, a target tooth is maintained in substantially fixed position with respect to a light source disposed within a lamp-head as shown, for example in FIG. 20.

As noted above, a spacer may be a light guide 1000, as shown in FIG. 11c, which includes an elliptically tubular member 1020 having an axial cavity 1040 disposed between a front aperture 1060 and a rear aperture 1080.

As shown in the illustrated embodiment, a first edge 1010 of the tubular member defines a substantially elliptically saddle shaped curve having a convex form in relation to a generally horizontal portion 1100 thereof and a concave form in relation to a generally vertical portion 1120 thereof. In addition, edge 1010 includes first and second substantially horizontal slots 1140, 1160. According to one embodiment of the invention, the slots 1140, 1160 are disposed substantially coplanar with respect to one another and are disposed substantially coincident with a major axis of the elliptically saddle shaped curve that defines edge 1010.

A rim 1180 extends radially inwardly from the edge 1010 to a second substantially elliptically saddle shaped curved edge 1200 (also referred to as the “second edge”) . The second edge 1200 is disposed in substantially constant spatial relation to edge 1010, whereby the rim 1180 has a substantially uniform radial dimension over the length of edge 1010. Edge 1200 defines an outer periphery of the front aperture 1060.

At the rear end of the embodiment of FIG. 11c, a third edge 1300 defines another curve that is of an approximately elliptically saddle shape. Edge 1300 is substantially concave in form in relation to a generally horizontal portion 1320. thereof and is generally convex in form in relation to a generally vertical portion 1340 thereof.

According to one embodiment of the invention, curve 1300 defines the rear aperture 1080 of the light guide.

According to one embodiment of the invention, the light guide does not include a rim adjacent the rear aperture 1080.

In one aspect of the illustrated embodiment, an outer surface 1390 of the light guide is disposed between edge 1010 and edge 1300. An inner surface 1360 of the light guide is disposed in a substantially uniform spatial relation to the outer surface 1390 so as to define inward and outward boundaries of the elliptically tubular member 1020.

In one embodiment of the invention, outer surface 1390 includes a plurality of gripping features 1380 adapted to improve the grip of an operator on surface 1390 during manipulation of the light guide 1000. In the illustrated embodiment, the gripping features 1380 have a raised elongated ellipsoid aspect. In another embodiment of the invention, the gripping features include a plurality of substantially hemispherical bumps. In still another embodiment of the invention, the gripping features include a plurality of zigzag grooves. One of skill in the art will appreciate that a wide variety of features may be disposed on surface 1340, so as to enhance overall gripability of the light guide 1000.

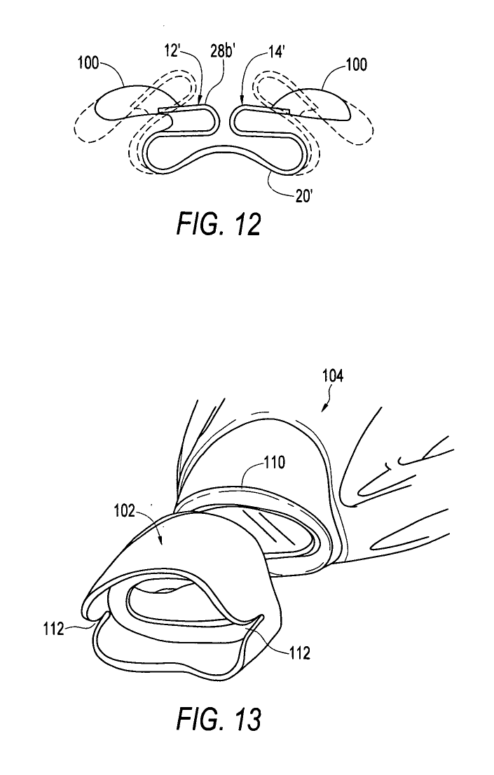

In FIG. 12, the lip retracting device 10′ is shown as it would appear inside a patient's mouth with all components located inside the patient's mouth except for the wing-like flanges 100 and the outside side walls 28b′, which would be outside the patient's mouth. As is readily apparent, the resilient member 20′ may be integrally molded, cast or attached to the inside side walls 28a′, to not interfere with the insertion of the lip retracting device 10′ into the mouth.

FIG. 13, shows a partial semi-schematic perspective view of a lamp 104 and a cone 102. The cone is configured to engage the nose opening 110 of the lamp 104. A pair of slots 112 is provided on the cone 102 for receiving the wing-like flanges 100. As may be visualized, when a patient is fitted with the lip retracting device 10′ and the wing-like flanges 100 engage the slots 112, the distance between the patient's mouth and the lamp 104 may be controlled. In addition, by securing the wing-like flanges 100 to the cone 102, movement by the patient during treatment may be minimized. As is readily apparent to a person of ordinary skill in the art, the size of the flanges 100, the depth of the slots 112, and the size of the cone 102 may vary to vary the distance between the patient and the lamp and the relative positioning of the patient relative to the lamp.

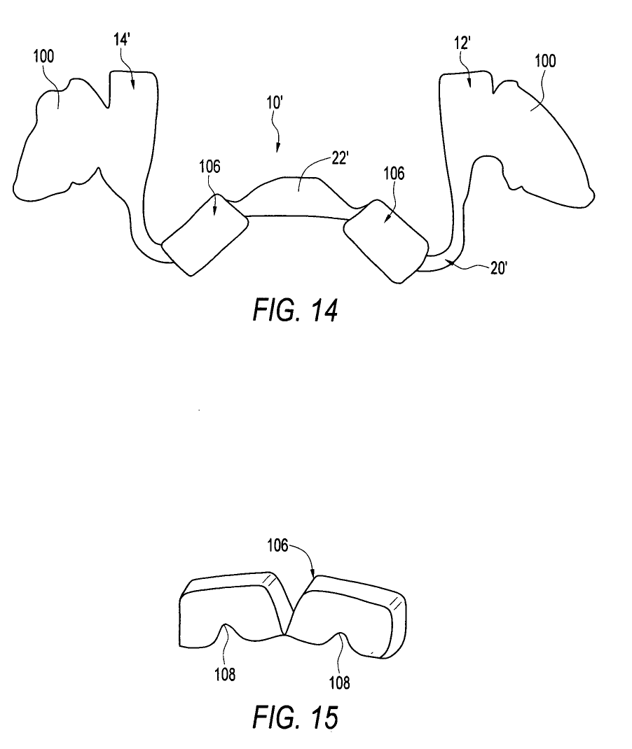

Pads may be provided with the resilient member 20′ to provide comfort for the patient when the patient is fitted with the lip retracting device 10′. The pads 106 may be positioned on the resilient member 20′, as shown in FIG. 14. The pads (FIG. 15) may be molded out of any rubber or foam. The rubber may be natural or synthetic. Synthetic rubbers may be, for example, elastomeric materials and may include, but not limited to, various copolymers or block copolymers(Kratons®) available from Kraton Polymers such as styrene-butadiene rubber or styrene isoprene rubber, EPDM (ethylene propylene diene monomer) rubber, nitrile (acrylonitrile butadiene) rubber, latex rubber and the like. Foam materials may be closed cell foams or open cell foams, and may include, but is not limited to, a polyolefin foam such as a polyethylene foam, a polypropylene foam, and a polybutylene foam; a polystyrene foam; a polyurethane foam; any elastomeric foam made from any elastomeric or rubber material mentioned above; or any biodegradable or biocompostable polyesters such as a polylactic acid resin (comprising L-lactic acid and D-lactic acid) and polyglycolic acid (PGA); polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers; and polyester/urethane resin.

The pad may be molded in two halves connected along one edge, like a clam-shell, as shown in FIG. 15. Each half may incorporate a slot, channel, or ridge 108 for receiving at least a portion of the resilient member 20′. The two halves may be fitted around the resilient member and then heat sealed together. Other methods of assembly that can achieve the same or similar results are also contemplated. For example, the ridges 108 may be so sized so that when the pad 106 is fitted over the resilient member 20′, the pad is able to slide relative to the resilient member to enable adjustment to the location of the pad on the resilient member.

For example, the pads 106 May be made of polyethylene closed-cell foam so that they may be sterilized. Open cell foams may also be used if they are amenable to autoclaving.

Also, the pads may have, for example, a smooth outside surface and smooth edges so that the pads may fit comfortably when in contact with the inside of a patient's mouth without unwanted irritation. In one embodiment, the pads may be designed to be placed over the second to the last molar when the lip retracting device 10′ is inside a patient's mouth. In addition, they may be made with, for example, relatively high resiliency material so that they, for example, may return to their original shape after use.

In another embodiment, the pads 106 may be made to be removable after each use. Here, the pads may be attached using removable adhesive or the two halves can simply be mated over the resilient member using detents or the like. Any foam material that may be made to fit comfortably inside a patient's mouth may be used as alternatives. The two-halves of the pad may also be made, for example, by heat set, to have a memory so that it may be forced open for installation onto a resilient member and be snapped shut when the opening force is removed. The two-halves may be integrally molded or attached along at least one side.

In a further embodiment, pads may be permanently affixed and may not be sterilizable, or autoclavable so that the retracting device may be a one-patient or one-use item, to be disposed of after use.

In yet a further embodiment, the pads may be an integral part of at least a portion of the resilient members 20. In this embodiment, at least one of the resilient members 20 may be formed in two portions connected by at least one pad. The resilient member may be constructed, for example, of polymeric or metallic material in wire-like configuration to minimize sharp edges.

Referring again to the resilient members 20 of FIG. 4, in one embodiment, the resilient members each may include a narrow mid-point 70 and two wide end points 72 relative to the mid-point. The end points 72 may also vary in widths or have the same width as compared to the other end points. In such an arrangement, the biasing force 52 (FIG. 3) of the resilient members 20 may be regulated by the width of the mid-point 70 relative to the end points 72. As is readily apparent to a person of ordinary skill in the art, the wider the mid-point 70, the more resistant it is to bending, which translates to more biasing force. Thus, the retracting force 52 of the lip retracting device 10 in retracting the cheeks and the lips can vary by varying the width of the mid-point 70. The retracting force may also be varied by changing the mechanical properties of the resilient member, such as introducing a mixture of polymers or other thermoplastics into the base material (i.e. a composite). In one embodiment, they may be made of materials having high modulus of elasticity.

In another embodiment of the present invention, a retracting device may include at least one non-inter-engaging formation. The non-inter-engaging formation may include a dental tray adapted for positioning a dental composition against a subject's teeth. In this embodiment, at least one inter-engaging formation, such as a wing-like member, may also be present for repeatably positioning a light source, or an imaging source to the subject undergoing dental procedure.

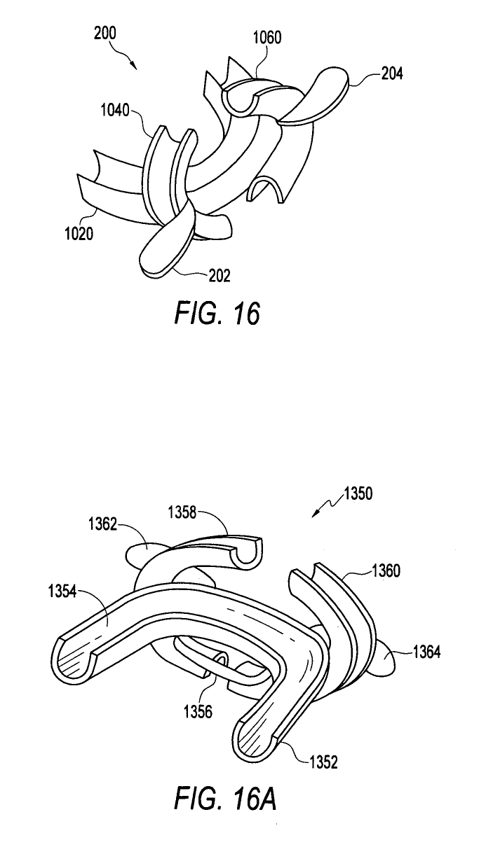

FIG. 16 shows an embodiment of a retracting device 200 of the present invention including a first u-shaped channel 1020 shaped and configured to accommodate a lower, or alternatively an upper, set of a patient's teeth (not shown). The lip retracting device 200 further includes a second u-shaped channel 1040 and a third u-shaped channel 1060 mounted substantially perpendicular to the first u-shaped channel 1020. The second and third u-shaped channels 1040, 1060 are adapted to receive the lips of the dental patient. The first u-shaped channel 1020 supports the second and third u shaped channels 1040, 1060 in substantially fixed spatial relation with respect to one another.

According to one embodiment of the invention, a pair of interface wings 202, 204 may be coupled to the second and the third u-shaped channels 1040, 1060 respectively. The interface wings 202, 204 may be shaped and configured to be received into slots 112 or 1130 and 1132 in a light guide 102 or 1120, fitted to a lamp head or light source 104 or 1102, as shown in FIGS. 13 and 20, respectively, in order to align the light source 104 or 1102 with the teeth of a patient. To the extent that a patient's lips effectively serve to couple a lamp head and teeth of the patient in fixed relation to the lip retracting device 10 or 1138, the lip retracting device 10 or 1138 is maintained in a substantially fixed position with respect to a light source disposed within a lamp-head as shown, for example in FIG. 20. This is described in greater detail below.

The interface wings or wing-like members, 202, 204, like the wing-like members of the above described lip retracting devices, typically have some rigidity so that the interface wings 202, 204 may form an effective interface when mated with the slots 112 of the light guide 102 or light source 104, as seen in FIG. 13. Similar to the embodiments described above, the interface wings 202, 204 may in a first arrangement be formed of the same material as the channels 1020, 1040, 1060. In a second arrangement, the interface wings 202, 204 are made of a different material from the channels 1020, 1040, 1060.

FIG. 16a shows the rear view of the embodiment of FIG. 16. The retracting device includes first and second lip receiving channels 1358, 1360. A third tooth receiving channel 1354 is disposed in a substantially normal orientation to the lip receiving channels and adapted to receive, within a concave region thereof, the upper or lower teeth of a patient. First and second formations such as wing-like members 1362, 1364 are coupled to the fist and second lip receiving channels 1358, 1360, respectively. As shown in the illustrated embodiment, a coupling member 1356 is disposed between, and mutually coupled to the first and second lip receiving channels 1358, 1360 and the tooth receiving channel 1352. One of skill in the art will appreciate that, in various embodiments, the retracting device includes a pair of teeth receiving channels arranged to receive both upper and lower teeth simultaneously.

In use, the retracting device 1350 is adapted to receive a dentistry composition, such as a dental whitening composition or a dental casting composition within the concave region 1354 of the tooth receiving channel 1352. The whitening composition may be light activatable or may be activatable in the dark. The teeth receiving channel may be transparent, translucent, or opaque, according to the desired application.

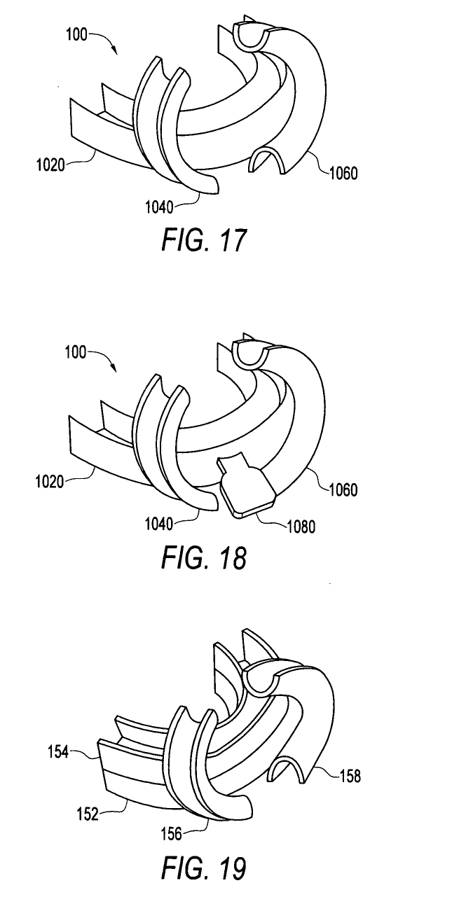

FIG. 17 shows a lip retracting device 1000 including a dental tray or teeth receiving channel according to one embodiment of the invention. The lip retracting device 1000 includes a first u-shaped channel 1020 shaped and configured to accommodate the lower, or alternatively the upper, set of a patient's teeth (not shown). The lip retracting device 1000 further includes a second u-shaped channel 1040 and a third u-shaped channel 1060 mounted substantially perpendicular to the first u-shaped channel 1020 and adapted to receive the lips of the dental patient and act as lip retracting devices holding the lips of the patient substantially clear of the teeth. The first u-shaped channel 1020 supports the second and third u-shaped channels 1040, 1060 in substantially fixed spatial relation with respect to one another.

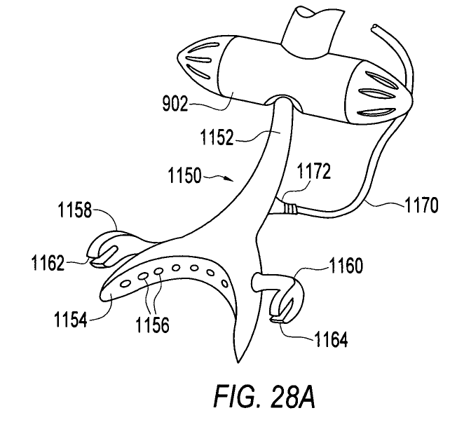

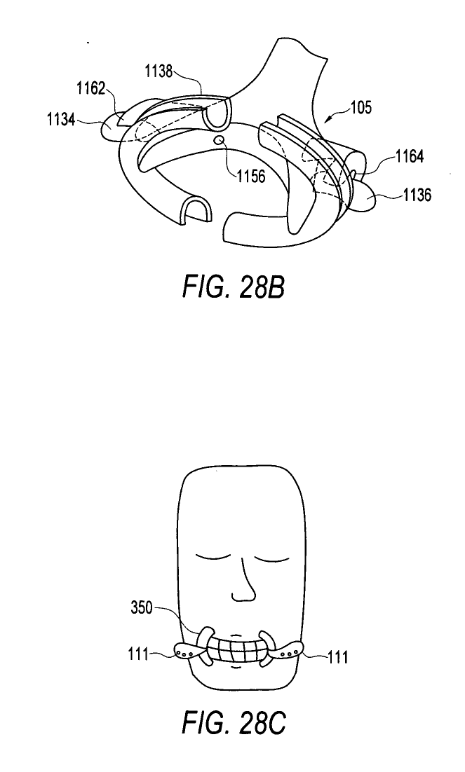

The first u-shaped channel 1020 may be configured again to receive a whitening composition. The first u-shaped channel 1020 may be fitted over a patient's teeth so that the whitening composition is in contact with the patient's teeth. The patient's lips in the second and third unshaped channels 1040, 1060 are held substantially clear of the teeth without effort on the part of the patient. In addition, the natural compression of the patient's lips may also maintain the lip retracting device in position without effort on the part of the patient, as is shown in FIG. 28c.

The lip retracting device 1000 may be flexible enough to be fitted to a patient and also rigid enough to support the whitening composition in place and to support the patient's lips. The lip retracting device 1000 may accordingly be made of any of the polymers mentioned above.

In an embodiment of the invention, the u-shaped channels 1020, 1040, 1060 are made of the same material. In a second embodiment of the invention, the first u-shaped channel 1020 is made of a different material from the second and third u-shaped channels 1040, 1060. In a first example arrangement, the first u-shaped channel 1020 may be made of rubber and the second and third u-shaped channels 1040, 1060 may be made of plastic. In a second example arrangement, the first u-shaped channel 1020 may be made of a UV-permeable material while the second and third u-shaped channels 1040, 1060 may be made of a material that is substantially impermeable to UV light. In this embodiment, a light-activated composition may be used because the first u-shaped channel 1020 allows penetration of UV light to whiten the teeth while the second and third u-shaped channels 1040, 1060 provide some protection for the patient's soft tissues.

In another alternative embodiment, the first u-shaped channel 1020 may be made as a custom fit for the patient's teeth. In this embodiment, an impression of the patient's teeth may be made and the first u-shaped channel 1020 is then manufactured using the impression.

In another alternative embodiment, the first u-shaped channel 1020 may be made of a material including a whitening compound. In a first arrangement, the included whitening compound may be activated by a substance applied to the patient's teeth before the lip retracting device is applied to the patient. In a second arrangement, the included whitening compound may be activated by the saliva of the dental patient. In this embodiment, typically the exterior of the first unshaped channel is covered with a layer of material that protects the patient's soft tissues. In a third arrangement, the included whitening compound is light-activated. In a fourth arrangement, the whitening composition may be activatable in the dark.

FIG. 18 shows a lip retracting device 100 having a first u-shaped channel 1020 mutually coupled to a second u-shaped channel 1040 and third u-shaped channel 1060 which are substantially perpendicular to the first u-shaped channel 1020. A tab 1080 is coupled to the first u-shaped channel 1020 between the second and third u-shaped channels 1040, 1060. The tab 1080 is useful for positioning the lip retracting device 1000 in the mouth of the dental patient. Later, after completion of the whitening process, the tab 1080 is useful for removing the lip retracting device 1000 from the mouth of the dental patient.

FIG. 19 is an alternative embodiment of the lip retracting device according to principles of the invention. The lip retracting device 150 includes a lower jaw u-shaped channel 152 and an upper jaw u-shaped channel 154. The lower jaw u-shaped channel 152 is shaped and configured to accommodate the lower set of the patient's teeth (not shown) while the upper jaw u-shaped channel 154 is shaped and configured to accommodate the upper set of the patient teeth (not shown). Both the lower jaw u-shaped channel 152 and the upper jaw u-shaped channel 154 are adapted to receive a treatment composition, such as a whitening composition. The lip retracting device 150 further includes a first u-shaped lip retracting device channel 156 and a second u-shaped lip retracting device channel 158 and they are mounted substantially perpendicular to the lower jaw u-shaped channel 152 and upper jaw u-shaped channel 154. The lower jaw u-shaped channel 152 and upper jaw u-shaped channel 154 support the a first u-shaped lip retracting device channel 156 and a second u-shaped lip retracting device channel 158 in substantially fixed spatial relation with respect to one another. The first and second u-shaped lip retracting device channels 156, 158 are shaped and configured to receive the lips of the dental patient.

In operation, the lower jaw u-shaped channel 152 and the upper jaw u-shaped channel 154 may each receive a whitening composition. The lower jaw u-shaped channel 152 and the upper jaw u-shaped channel 154 may then be fitted over a patient's teeth so that the treatment composition such as a whitening composition is in contact with the patient's teeth. The patient's lips are received into the first and second u-shaped lip retracting device channels 156, 158. Using the dental lip retracting device of the present embodiment, a dental process such as a whitening process may be performed on the lower teeth and the upper teeth at the same time effectively reducing the overall duration of the session.

FIG. 20 shows an assembly relationship between the inter-engaging formations of the light system 1102, the light guide 1120 and the lip retracting device 1122 according to one embodiment of the invention. One of the formations, for example, a pivot mount 906, couples another formation, such as a ball joint 902 to a lamp head 1102. The ball joint 902 allows the lamp head 1102 to be swiveled in space such that an optical axis of the lamp system may be aligned with a target tooth of, for example, a dental whitening subject.

A light guide 1120 having at least one formation adapted to be coupled to an anterior end of the lamp head 1102, by means of another formation. In one embodiment, the light guide 1120 includes an inner surface region 1122 that is adapted to be held in proximity to an outer surface region 1124 of the lamp head 1102. According to one embodiment of the invention, a formation such as a projecting member, or bump, on inner surface 1122 is adapted to be urged into a formation, such as a recessed region 1126 of outer surface region 1124.

In one embodiment of the invention, the light guide 1120 includes an elastically compressible cushion 1128 at an anterior edge thereof. The elastically compressible cushion 1128 may serve to soften an interface between a dental whitening process subject (not shown) and the light guide 1120.

In a further aspect of the invention, as shown in the illustrated embodiment, the light guide 1120 includes formations such as first and second slots 1130 and 1132. These slots may be adapted to receive formations, such as projecting wings 1134, 1136 of a lip retracting device 1138 so as to stabilize a relationship between, for example, the dental whitening subject and the lamp head 1102.

The lip retracting device 1138 includes channels 1140, 1142 adapted to support the lips of a dental whitening subject during the whitening or other dental process, and an elastic member 1144. The elastic or elastomeric member 1144 is coupled to the channels 1140, 1142 and adapted to urge the channels outwardly towards the lips, so as to couple the subject undergoing the dental process to the lip retracting device.

When the subject is coupled to the lip retracting device 1138, and the lip retracting device is coupled to the light guide 1120 by the insertion of wing-like members 1134, 1136 in the respective slots 1130, 1132 in the light guide 1120, the subject is spatially stabilized with respect to the lamp head 1102. In this way the support structure serves to support the lamp head in a substantially stable spatial relationship to the, for example, whitening subject.

As discussed above, this spatially stabilized relationship between a subject and the support structure of the invention is found in other embodiments of the invention and in relation to various apparatus and processes.

According to another embodiment of the invention, the light guide 1120 may include one or more ventilation holes (not shown) adapted to allow a patient to breathe more easily during use of the light guide apparatus.

In one embodiment the elastic compressible member 1128 is, for example, made from polymer foam or from rubber and is attached to the light guide 1120 by heat sealing or by an adhesive. Suitable adhesives can include, but are not limited to, hot melt adhesives, pressure sensitive adhesives, reactive adhesives or the like. Alternatively, suitable adhesives can be acrylic-based, polyurethane-based, epoxy-based, polyamide-based, cyanoacrylate-based, styrene copolymer-based, polyolefin-based or similar. Further, the elastic compressible member 1128, which may be present in pieces, for example, 1128a, 1128b, may be integrally molded onto the light guide.

In the present embodiment, the cushioning elastic member 1128 may be made in two pieces, an upper portion 1128a and a lower portion 1128b, extending the slots 1130, 1132 of the light guide so as to accommodate wing members of an exemplary lip retracting device.

The elastic cushioning member 1128 of the light guide, or the elastic member 1144 of the lip retracting device, may be made of any foam material, which may be either an open cell or closed cell foam, including those described above that are useful for pads for the lip retracting device.

The air vents may be configured to pass air but still prevent light leakage out of the light guide 1120.

According to one embodiment of the invention, a light guide 1120, as shown in FIG. 20, may be configured as a single-use item, used for one dental whitening treatment and then discarded. With this in mind, as discussed above, the light guide 1120 further includes a memory integrated circuit 2120 disposed within a space 2040 molded into the underside of the light guide 1120. The memory integrated circuit 2120 stores a record of a duration of use signal indicating how long the particular light guide has been in use, as seen in FIG. 25. The light guide memory integrated circuit 2120 may be part of a system for ensuring that the light guide 1120 is not improperly reused. The light guide 1120 may also be configured to be a one-patient use item by having an embedded memory chip.

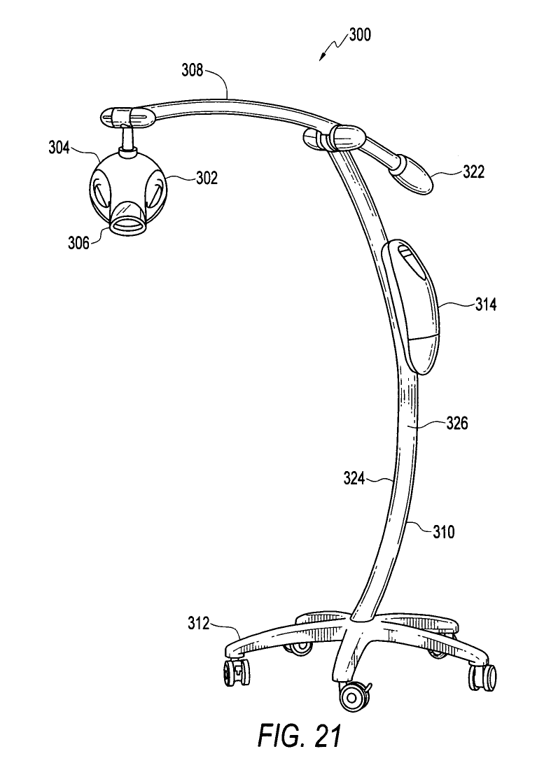

FIG. 21 shows one embodiment of, for example, a dental whitening system suitable for use with embodiments of the present invention. A perspective view of a dental whitening lamp system 300 includes a lamp head 302 having a lamp head housing 304 and a light guide 306. The lamp head 302 provides the light that activates a whitening or filling substance applied to a patient's teeth by generating light at the lamp head housing 304 and directing the light through the light guide 306. This lamp system 300 can be used in a dental office or a dental laboratory. The lamp housing may also include at least one heat sink, in the proximity of the light source for keeping the light source and the lamp housing cool. The heat sink may be made of any material that has good thermal conductivity, including metal blocks of copper, aluminum or similar. In another embodiment, the cooling system includes heat pipes. In another embodiment, the cooling system includes phase change materials, some embodiments and material are exemplified as is described in U.S. Application No. 60/585,224, “Dental Light Devices With Phase Change Material Filled Heat Sink”, filed on Jul. 2, 2004, the contents of which are incorporated herein by reference.

The heat sink may be constructed by hollowing out a thermally conductive material, such as metal, and at least partially filling the void with at least one phase change material prior to capping it to secure the phase change material inside, such that the at least one phase change material is substantially contained or surrounded by a thermally conductive material such as metal normally used in the construction of a conventional heat sink.

Alternatively, the heat sink may be cast or machined from a thermally conductive material, such as metal, to create walls surrounding a bore or void. The bore or void is partially filled with at least one phase change material prior to capping it to secure the material inside.

In one embodiment, the inventive heat sink may be used by itself. In another embodiment, it may be used in addition to a fan, in conjunction with a conventional metal block heat sink or combinations thereof.

Suitable phase change material may include organic materials, inorganic materials and combinations thereof. These materials can undergo substantially reversible phase changes, and can typically go through a large, if not an infinite number of cycles without losing their effectiveness. Organic phase change materials include paraffin waxes, 2,2-dimethyl-n-docosane (C₂₄H₅₀) , trimyristin, ((C₁₃H₂₇COO)₃C₃H₃), and 1,3-methyl pentacosane (C₂₆H₅₄). Inorganic materials such as hydrated salts including sodium hydrogen phosphate dodecahydrate (Na₂HPO₄.12 H₂O), sodium sulfate decahydrate (Na₂SO₄.10H₂O), ferric chloride hexahydrate (FeCl₃.6 H₂O), and TH29 (a hydrated salt having a melting temperature of 29° C., available from TEAP Energy of Wangara, Australia) or metallic alloys, such as Ostalloy 117 or UM47 (available from Umicore Electro-Optic Materials) are also contemplated. Exemplary materials are solids at ambient temperature, having melting points between about 30° C. and about 50° C., more for example, between about 35° C. and about 45° C. Also, the exemplary materials have a high specific heat, for example, at least about 1.7, more for example, at least about 1.9, when they are in the state at ambient temperature. In addition, the phase change materials may, for example, have a specific heat of at least about 1.5, more for example, at least about 1.6, when they are in the state at the elevated temperatures.

The phase change material may also have a high latent heat of fusion for storing significant amounts of heat energy. This latent heat of fusion may be, for example, at least about 30 kJ/kg, more for example, at least about 200 kJ/kg.

Thermal conductivity of the materials is a factor in determining the rate of heat transfer from the thermally conductive casing to the phase change material and vice versa. The thermal conductivity of the phase change material may be, for example, at least about 0.5 W/m° C. in the state at ambient temperature and at least about 0.45 W/m° C. in the state at elevated temperature.

In general, the phase change material may be contained inside a thermally conductive material, such as a metal casing. The casing defines a bore, which may be of any shape, but is for example, generally of a cylindrical or rectangular shape. The metal casing or wall of the bore acts to contain the phase change material, and to also aid in conducting heat to and away from the phase change material. The thinner the wall, the more phase change material can be present in a given size of the heat sink, and the less it contributes to the weight of, for example, the curing light. However, the thinner the wall, the less efficient the heat sink maybe in conducting heat away from the phase change material and the longer it will take to return the phase change material to ambient temperature and its original state, so that it may function as a heat sink again. For example, the wall thickness ranges from about 1 mm to about 2.5 mm, more for example, from about 1 mm to about 1.5 mm.

The casing may also be constructed to have a large surface area. A structure having fins or other features that serve to increase the surface area for heat conduction or convection is desirable, thus a spherical structure, though useful, is not the optimal choice. Such fins or other surface area increasing features may also be incorporated into the bore to increase the contact area between the thermally conductive casing and the phase change material, thus permitting faster more efficient transfer of heat between the thermally conductive casing and the phase change material.

The thermally conductive casing can also provide a good thermal contact for heat transfer from the light source. This may be accomplished with a smooth, thermally conductive surface with a high area of contact. Also, thermal coupling may be achieved with thermally conductive interface materials such as thermal epoxy. Interface materials that are electrically insulating are also useful in isolating the light source from the heat sink in an electrical sense without losing thermal conductivity.

The lamp head 302 may be attached to a first end of a boom 308. The lamp head 302 is positionable with respect to the boom 308 and ay have a wide range of motion with respect to the end of the boom 308. The boom 308 is supported by a mast 310. In the illustrated embodiment, the boom 308 is pivotally mounted to the mast 310 at a point on the boom 308 closer to a second end of the boom 308 than the lamp head housing 304.

The boom 308 is adjustably positionable with respect to the mast 310. The boom 308 has both a rotational and a tilt range of motion with respect to the mast 310. A counterweight 322 on the second end of the boom 308 provides a counterbalance for the lamp head 302. The mast 310 is attached to the base 312. In the embodiment shown, base 312 is a rolling base.

The rolling base 312 enables the entire lamp system 300 to be positionable with regard to a patient in a dental chair. In addition, the mast 310 in other embodiments of the invention may be axially rotatable with respect to the base 312. According to one embodiment of the invention the mast 310 is curved and the curve accordingly defines a concave side 324 and a convex side 326 of the mast 310. In the embodiment illustrated, a power pack 314 is attached to the mast 310 on the convex side 326. The power pack 314 includes controls for the lamp system 300.

In operation, the lamp system 300 may be positioned with respect to the patient in a dental chair (not shown). Once the lamp system 300 is positioned with respect to the patient, the operator may align the light guide 306 with the patient's mouth, as discussed above. In an alternative embodiment, the light guide 306 may be an integral part of the lamp head 302.

The light guide 306 may be set to a wide range of positions through the wide range of motion of both the boom 308 with respect to the mast 310 and the lamp head 302 with respect to the boom 308. The light guide 306 may be shaped and configured to mate with a lip retracting device with interface wings, such as the lip retracting device shown in FIG. 16, worn by the patient, thereby providing a substantially precise alignment with the patient's mouth.

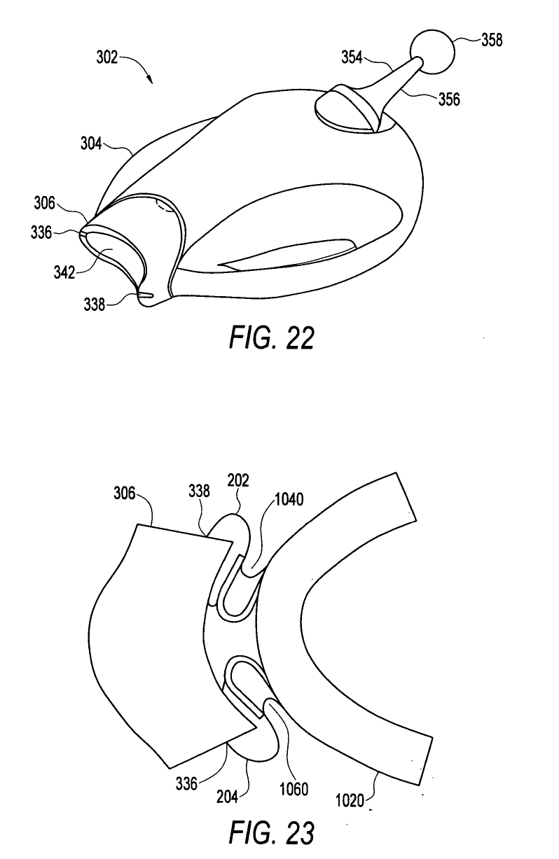

FIG. 22 is a perspective view of the lamp head 302 of FIG. 21 and suitable for use with the present invention. The lamp head 302 includes the lamp head housing 304 and the light guide 306, as noted above. The lamp head housing 304 produces and directs light through the light guide 306. In the present embodiment, the lamp head housing 304 produces light with a light source, such as a bulb. Other types of light sources are possible within the scope of the invention, including a lamp, an arc lamp such as a halogen light source, semiconductor light emitting devices, light-emitting chips such as an LED, a solid state LED, an LED array, a fluorescent bulb, and so on. In the case of dental imaging, the light source may include the above in addition to a laser, an x-ray or even an infrared source. The light guide 306 may serve as an interface between the lamp system 300 and the patient having, for example, a dental whitening treatment.

The light guide 306, according to one embodiment of the invention, acts as an interface between the lamp system 300 and the subject receiving dental whitening treatment. The structure of the light guide 306 includes a UV-inhibiting material in order to protect the patient's skin from ultra-violet light exposure. The light guide 306 is generally shaped like an ellipsoidal tube. A first slot 336 and a second slot 338 cut on opposing sides of a front opening 342 of the light guide 306 align with the interface wings 202, 204 on the lip retracting device 200, as shown in FIG. 23, to align the lamp head 302 accurately with the patient.

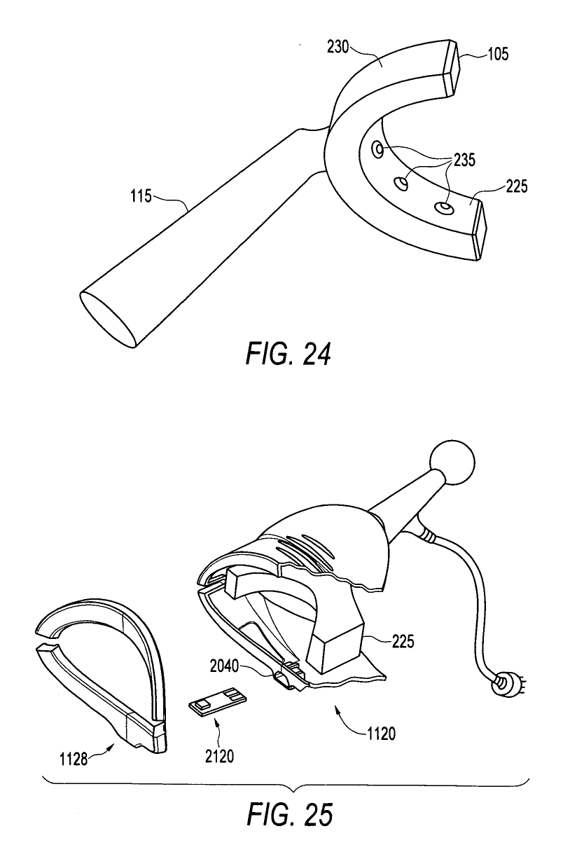

In another embodiment, multiple light sources arranged in a geometric arrangement in an illumination frame may be used in the illumination system. In one embodiment, the light sources may be arranged in an arcuate form and may, for example, conform to the jaw of a patient, as shown in FIG. 24.

In FIG. 24, the illumination frame 105 has an arcuate shape and is configured to provide illumination to teeth. The arcuate shape follows the curvature of the human jaw (and human head) so that the light sources are approximately equidistant from the various teeth toward which the light sources are directed. The illumination frame 105 has a front 225 and a back 230. The front 225 is concave and the back 230 is convex. The tube 115 is attached to the back 230 of the illumination frame 105. The tube 115 provides support for the illumination frame 105 and also acts as a conduit for wiring for the illumination frame 105. A plurality of light sources 235 is arranged along the front 225 of the illumination frame 105. The light sources can be any of the ones mentioned above, but are not limited to them. The light sources 235 generate and direct light toward the patient's teeth (not shown) for a whitening or a curing process, for example. In a first embodiment, the light sources 235 emit light having substantially the same light spectrum. In a second embodiment, the light sources 235 emit light having different spectra. In one embodiment of the invention, the light sources 235 protrude from the surface of the front of the illumination frame 225. In an alternative embodiment, the light sources 235 are disposed substantially flush with the surface of the front of the illumination frame 225.

In the illumination system with multiple light sources, the light sources may be collectively powered or individually powered. If individually powered, each of the individual light sources may be turned on or off separately, as desired. This is especially useful for a curing or imaging process, where only one or two teeth may be undergoing treatment or being examined.

In one aspect, the illumination frame 225 may be disposed inside a light guide housing, as shown in FIG. 25. Just as described and shown in FIG. 20 above, an elastic member 1128 is disposed between the patient and the light guide 1120. The elastic member 1128 serves to cushion the interface between the patient and the light guide, absorbing shocks which might otherwise be painful or uncomfortable.

The light guide 1120 may be mated to the lip retracting device 1138, similar to that described above in relationship to FIG. 20.

In another aspect, the illumination frame may be in place of the lamp head housing as a self-contained structure, such as shown in FIGS. 24, 25 and 26. In FIG. 26, the illumination frame 105 has a generally arcuate shape having a first end 500 and a second end 505. The back 230 of the illumination frame 105 is also convex and the front 225 of the illumination frame 105 is concave. Each end 500, 505 has a slot 510, 515 open from the front 225 of the illumination frame 105 towards the back 230 of the illumination frame 105. Each slot 510, 515, extends inwardly from its respective end 500, 505 of the illumination frame 105. The slots 510, 515 are located and configured to mate with the wings of a lip retracting device as described above.

FIG. 25a shows a top view of an illumination frame 105 including a heat sink according to one embodiment of the invention. The illumination frame 105 has a plurality of light sources 235, having a heat sink 650 coupled to their ballasts (or, base). The heat sink 650 may be made of any material as described above, including a phase change material. The heat sink may also be of any shape.

FIG. 28 shows a top view of a lip retracting device 350 mated to an illumination frame 105 according to one embodiment of the invention. The lip retracting device 350 acts as a fixturing device for maintaining the illumination frame 105 in substantially fixed relation with respect to a target tooth. As shown in the illustrated embodiment, the lip retracting device 350 includes first 355 and second 360 U shaped channels adapted to receive the lips of a dental patient adjacent to respected internal surfaces 365, 370 thereof.

A resilient or support member 375 is mutually coupled to the U shaped channels 355, 360 and adapted to support the U shaped channels 355, 360 in substantially fixed spatial relation with respect to one another. According to one embodiment of the invention (not shown) the support member 375 also supports a tongue-cap adapted to retain a patient's tongue and shield the same from incident illumination.

According to one embodiment of the invention, a pair of interface wings 380, 385 may be coupled to the U shaped channels 355, 360 respectively. According to one embodiment of the invention, the interface wings 380, 385 each include a respective upper surface 390, 395 and a respective lower surface (not shown). According to one embodiment of the invention, upper surfaces 390 and 395 are disposed substantially parallel in relation to lower surfaces. Pursuant to one embodiment of the invention, the interface wings 380, 385 have a first plurality of ticks or holes 410, 415 respectively. The ticks or holes are adapted to facilitate maintaining a particular alignment of the illumination frame (as shown in FIG. 26) 105 with respect to the lip retracting device 350, as discussed above.

According to one embodiment of the invention, interface wings 380, 385 are adapted to be received within slots 310, 315 (as shown in FIG. 28) respectively. By pressing the lip retracting device 350 toward the first end 500 and the second end 505 of the illumination frame 105, the interface wings 380, 385 are urged into slots 510, 515, whereby the orientation and position of the lip retracting device 350 with respect to the illumination frame 105 is substantially fixed. Consequently, to the extent that a patient's lips effectively serve to couple the head and teeth of the patient in fixed relation to the lip retracting device 350, a target tooth is maintained in substantially fixed position with respect to light sources 235 disposed at the front 225 of the illumination frame 105.

In operation, the support member 375 is disposed inside the mouth of the patient. The patient's lips are held apart by the U-shaped channels 355, 360. The interface wings 380, 385 are pressed into the slots (not shown here, but are shown in FIG. 26 as 510 and 515) of the illumination frame 105 effectively holding the illumination frame 105 in place in proximity to the teeth of the patient. In addition, the holes 410, 415 enable the mating of the illumination frame 105 with the lip retracting device 350 to be adjusted according to the patient. In an alternative embodiment of the invention, the illumination frame 105 and lip retracting device 350 are fixedly attached to each other. The illumination frame in this embodiment is substantially permanently aligned with the lip retracting device as one integral part.

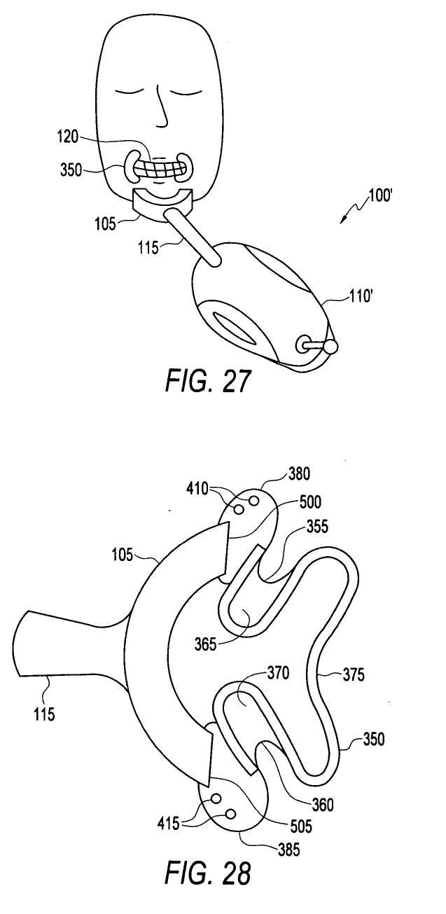

In a further aspect, the illumination frame may be in addition to the lamp head housing, as shown in FIG. 27. The dental illumination system 100′ includes an illumination frame 105 connected to a lamp head 110′ by a tube 115. The illumination frame 105 provides light to activate a whitening substance or curing composite applied to a patient's teeth 120. The patient typically wears a lip retracting device 350. The illumination frame 105 and lamp head 110′ together generate and direct light toward the patient's teeth 120 for a whitening or a curing process, for example. In one alternative embodiment, the illumination frame 105 and tube 115 is adjustable with respect to the lamp head 110′. In another alternative embodiment, the tube 115 is flexible and can be adjusted to various positions. In another embodiment, the illumination frame 110′ is flexible.

In the illumination system with multiple light sources, the light sources may be collectively powered or individually powered. If individually powered, each of the individual light sources may be turned on or off separately, as desired. This is especially useful for a curing process, where only one or two teeth may be undergoing treatment.

FIG. 28a shows a dental illumination frame according to another embodiment of the invention. In the illustrated embodiment, the dental illumination source may be a dental whitening illumination source, a dental composition curing source or an imaging head. In the illustrated embodiment, the ball joint 902 is coupled to a light housing 1150. The light housing 1150 includes a first elongate portion 1152 having at its posterior end the ball of the ball and socket joint 902. An anterior end of the housing 1150 includes, in the illustrated embodiment, an arcuate surface 1154.

Arcuate surface 1154 supports one or more illumination sources 1156. In one embodiment of the invention the one or more illumination sources 1156 includes one or more optical fibers coupled to a remote light source and/or one or more optical wavelength transformer such as those described in U.S. patent application No. 60/658,517, the disclosure of which is herewith incorporated by reference in its entirety.

In the illustrated embodiment, a signal cable 1170 is coupled at one end to the light housing 1150. The signal cable may include a power cable adapted to provide power for the one or more illumination sources 1156. The single cable may also include an optical light guide such as an optical fiber adapted to transmit light to the one or more illumination sources from a remote light source. In at least one embodiment of the invention, the signal cable 1170 includes a strain-relief feature 1172.

Illustrating another aspect of the invention, the embodiment of FIG. 28a shows first and second slot 1162, 1164. The slots 1162, 1164 are adapted to receive corresponding wings 1134, 1136 of a lip retracting device 1138, as illustrated in FIG. 28b.

FIG. 28b shows a view of an illumination frame 105 fitted with a retracting device 1138 worn by a dental procedure subject. Insertion of the wings 1134, 1136 into the slots 1162, 1164 serves to stabilize a spatial relationship between the subject and the one or more illumination sources 1156.

FIG. 28c shows a front view of a lip retracting device 350 of FIG. 11a being worn by a person. The interface wings 111 are located at either side of the lip retracting device 350 and are available for mating with the imaging head, such as a light source 102 as shown in FIG. 20.

In yet another embodiment of the present invention, a retracting device may include at least one non-inter-engaging formation. The non-inter-engaging formation may include a film holder. The device may also include at least one inter-engaging formation such as a wing-like member for repeatably positioning a light source or an imaging source to a subject undergoing dental procedure.

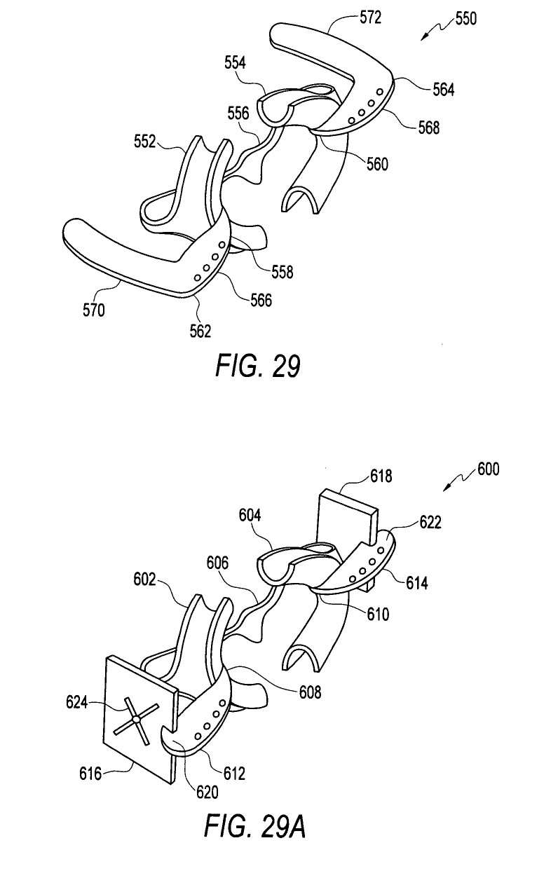

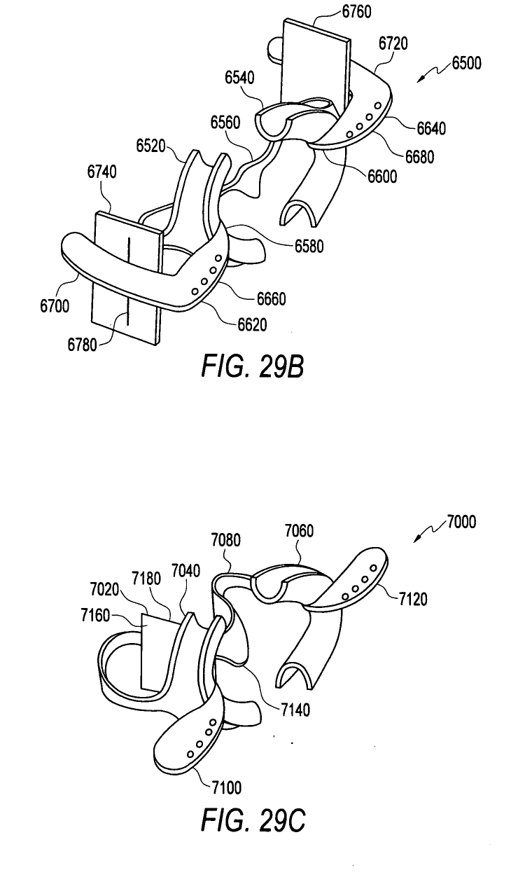

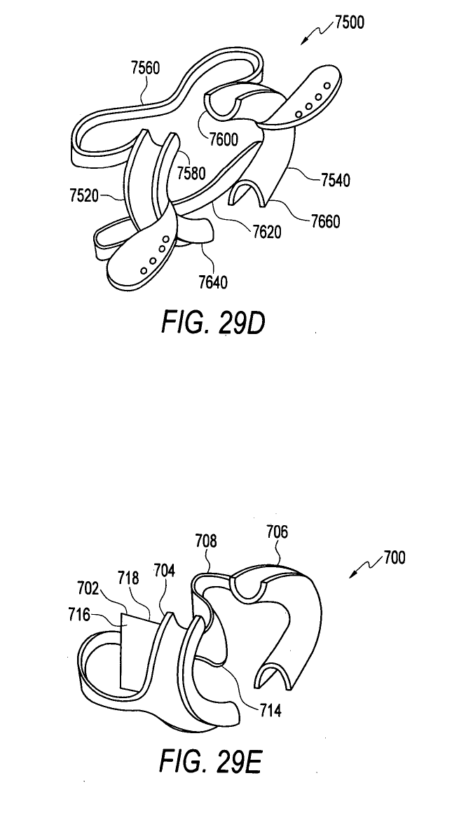

FIG. 29 shows a lip retracting device 550 according to an alternative embodiment of the invention. The lip retracting device 550 has a first u-shaped channel 552 and a second u-shaped channel 554 to hold the lips of the patient whose teeth are to be imaged. A support member 556 is mutually coupled to the u-shaped channels 552, 554 and is adapted to support the unshaped channels 552, 554 in substantially fixed spatial relation with respect to one another. A first end 558 of the first interface wing 562 is coupled to the first u-shaped channel 552. A first end 560 of the second interface wing 564 is coupled to the second u-shaped channel 554.

Each of the interface wings 562, 564 includes a first portion 566, 568 located at the front of the lip retracting device 550 away from the patient. A second portion 570, 572 of each interface wing 562, 564 extends outward and toward the back of the lip retracting device 550. In operation, the first portions 566, 568 are located at the front of the patient's face while each second portion 570, 572 is located at a side of the patient's face. The interface wings 562, 564 are adapted to be received within the slots 236, 238 of the beam guide 106. The first portions 566, 568 enable the beam guide 106 to be aligned to the front of the patient. The second portions 570, 572 of the interface wings 562, 564 enable the beam guide 106 and imaging head 102 to be aligned on either side of the patient's head.

FIG. 29a shows a lip retracting device 600 according to an alternative embodiment of the invention. The lip retracting device 600 includes a first u-shaped channel 602 and a second u-shaped channel 604 to hold the lips of the patient whose teeth are to be imaged. A resilient or support member 606 is mutually coupled to the u-shaped channels 602, 604 and is adapted to support the u-shaped channels 602, 604 in substantially fixed spatial relation with respect to one another. A first end 608 of the first interface wing 612 is coupled to the first u-shaped channel 602. A first end 610 of the second interface wing 614 is coupled to the second u-shaped channel 604. The interface wings 612, 614 are adapted to be received within the slots 236, 238 respectively of the beam guide 106. A target 616, 618 is coupled to a second end 620, 622 of the first interface wing 612 and second interface wing 614 respectively. Each of the targets 616, 618 has alignment markings 624. In operation, the targets 616, 618 act as visual alignment mechanisms for the imaging head 102 for side images of the patient's teeth.

FIG. 29b shows a lip retracting device 6500 according to an alternative embodiment of the invention. The lip retracting device 6500 has a first u-shaped channel 6520 and a second u-shaped channel 6540 to hold the lips of the patient whose teeth are to be imaged. A resilient or support member 6560 is mutually coupled to the u-shaped channels 6520, 6540 and is adapted to support the u-shaped channels 6520, 6540 in substantially fixed spatial relation with respect to one another.

A first end 658 of the first interface wing 6620 is coupled to the first u-shaped channel 6520. A first end 6600 of the second interface wing 664 is coupled to the second u-shaped channel 6540. Each interface wing 6620, 6640 includes a first portion 6660, 6680 located at the front of the lip retracting device 6500 away from the patient. A second portion 5700, 5720 of each interface wing 6620, 6640 extends outward and toward the back of the lip retracting device 6500. In operation, the first portions 6660, 6680 are located at the front of the patient's face while each second portion 6700, 6720 is located at a side of the patient's face. The interface wings 6620, 6640 are adapted to be received within the slots 236, 238 of the beam guide 106. The first portions 6660, 6680 enable the beam guide 106 to be aligned to the front of the patient. The second portions 6700, 6720 of the interface wings 6620, 6640 enable the beam guide 106 and imaging head 102 to be aligned on either side of the patient's head.

A target 6740, 6760 is coupled to the second portions 6700, 6720 of the first interface wing 6620 and second interface wing 6640 respectively. Each target 6740, 6760 has visual alignment markings 6780. In operation, the targets 6740, 6760 act as visual alignment mechanisms for the imaging head 102 for side images of the patient.

FIG. 29c shows a lip retracting device 7000 according to one embodiment of the invention. The lip retracting device 7000 is also a fixturing device for maintaining the imaging head 1102 (as shown in FIG. 20) in substantially fixed relation with respect to a target tooth. Further, the lip retracting device 7000 holds film inside the teeth of the patient in a manner in which the patient does not actively participate in the holding. In bite-wing images, for example, the patient bites down on a portion of the film holder in order to maintain the imaging film in a proper position. For people with poor muscle control, biting down for even a short period of time may be difficult. A film holder 7020 attached to the lip retracting device 7000 as described below does not require the active participation of the patient to hold the film in place. p As shown in the illustrated embodiment, the lip retracting device 7000 includes first 7040 and second 7060 semicircular “U”-shaped channels adapted to receive the lips of a dental patient as described above with respect to FIG. 11a. A support member 7080 is mutually coupled to the “U”-shaped channels 7040, 7060 and adapted to support the “U”-shaped channels 7040, 7060 in substantially fixed spatial relation with respect to one another. According to one embodiment of the invention, a pair of interface wings 7100, 7120 is coupled to the “U”-shaped channels 7040, 7060 respectively. According to one embodiment of the invention, interface wings 7100, 7120 are adapted to be received within slots 1130 and 1132 respectively (as shown in FIG. 20).

The support member 7080 is shaped and configured to support a film holder 7020. In one embodiment, the film holder 7020 is an integral part of the lip retracting device 7000. In an alternative embodiment, the film holder 7020 is separably coupled to the lip retracting device 7000. With the lip retracting device 7000 held in place by the patient's lips in the u-shaped channels 7040, 7060, the film in the film holder 7020 is also held in place. The lip retracting device 7000 is symmetric and accordingly the film holder could be located on either side of the central portion 7140 of the support member 7080. In an alternative embodiment of the invention, the support member 7080 may be configured to support two film holders 7020, one on either side of the central portion 7140 of the support member 7080. In this embodiment, the front 7160 of the film holder 7020 is x-ray penetrable while the back 7180 of the film holder 7020 blocks x-rays. In a first arrangement, the front 7160 of the film holder 7020 may be made of an x-ray penetrable material while the back 7180 of the film holder 7020 may be made of an x-ray blocking material. In a second arrangement, an x-ray blocking lining is inserted behind the film in the film holder 7020.

FIG. 29d shows a lip retracting device 7500 according to one embodiment of the invention. The lip retracting device 7500 is also a fixturing device for maintaining the imaging head 102 in substantially fixed relation with respect to a target tooth. Further, the lip retracting device 7500 improves access to the patient's mouth so that the film may be inserted and removed easily in order to make a series of images.

As shown in the illustrated embodiment, the lip retracting device 7500 includes first 7520 and second 7540 semicircular “U”-shaped channels adapted to receive the lips of a dental patient. A first support member 7560 is mutually coupled to first ends 7580, 7600 of the u-shaped channels 7520, 7540. A second support member 7620 is mutually coupled to second ends 7640, 7660 of the u-shaped channels 7520, 7540. The support members 7560, 7620 are adapted to support the u-shaped channels 7520, 7540 in substantially fixed spatial relation with respect to one another. The support members 7560, 7620 leave a space between them that enables the film to be inserted into the patient's mouth for imaging purposes while the u-shaped channels 7520, 7540 hold the patient's lips clear.

In the present embodiment of the invention, a pair of interface wings 7680, 7700 are coupled to the “U”-shaped channels 7520, 7540. The interface wings 7680, 7700 are adapted to be received within slots 1140, 1160 respectively of the beam guide 1000 (shown in FIG. 11c) where the imaging head 102 is located at the front of the patient. In an alternative embodiment, the interface wings 7680, 7700 are extended as in the embodiment illustrated in FIG. 29. The extended interface wings enable the imaging head 102 to be positioned at either side of the patient's head.

FIG. 29e shows an alternate embodiment of FIG. 29c where only non-inter-engaging formations are included for positioning an imaging film or sensor adapted for imaging.

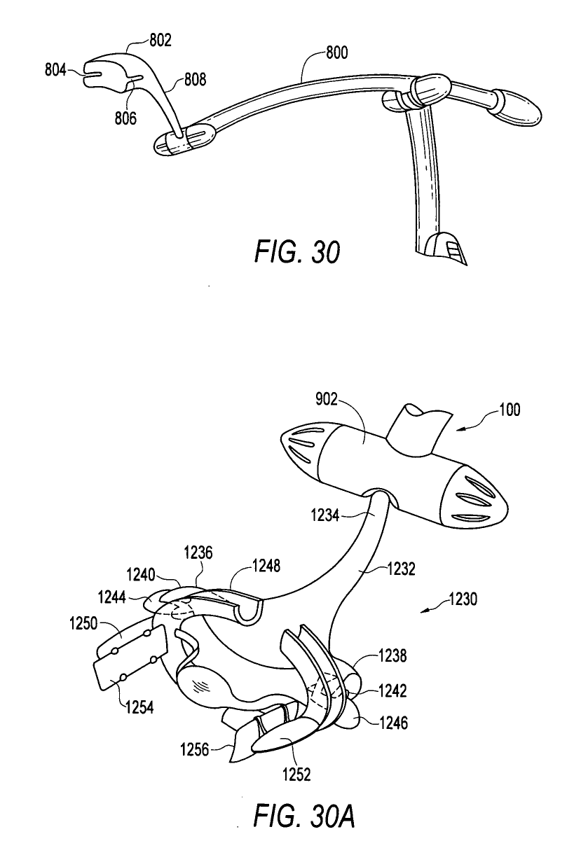

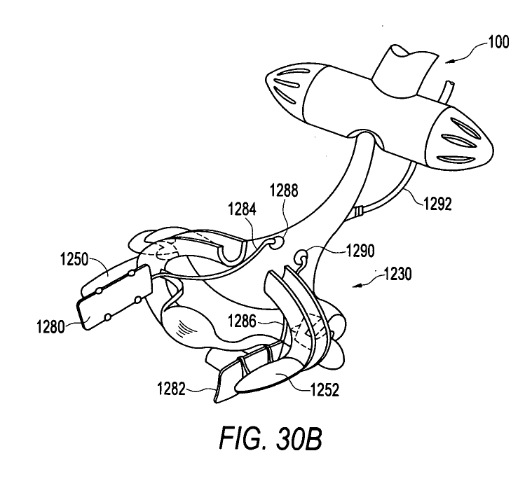

FIG. 30 shows shows an imaging support stand 800 suitable for use with certain types of imaging apparatus, such as panoramic x-ray devices. A patient to be imaged holds a position in relation to the support stand 800 while an imaging device (not shown) travels around the patient's head. The support stand 800 includes a coupling device 802 that enables the patient to hold the position.

The coupling device 802 is attached to the imaging support stand 800 by a support member 808. The support member 808 is coupled to the coupling device 802 and the imaging support stand 800. The support member 808 is, in one embodiment, adjustably coupled to the support stand 800 by a ball and socket joint that enables desired positioning of the coupling device.

The coupling device 802 is similar to the slotted beam guide 106, as shown for example in FIG. 11c. The coupling device 802 is shaped and configured to mate with a lip retracting device with interface wings such as the lip retracting device 350 with interface wings 111 shown in FIG. 11a. The coupling device 802 has a first slot 804 and a second slot 806 adapted to couple with interface wings 111. By pressing the lip retracting device 350 toward the front edge of the coupling device 802, the interface wings 111 are urged into the slots 804, 806 whereby the orientation and position of the lip retracting device 350 is substantially fixed. Accordingly, the orientation and position of the patient's head is also fixed.

FIG. 30a shows another embodiment of the invention including a dental support structure 100 that is adapted to support a dental imaging fixturing system 1230. In the illustrated embodiment, the ball joint 902 is coupled to a support member 1232. The support member 1232 includes a first elongate portion 1234 having at its posterior end the ball of the ball and socket joint 902. An anterior end of the support member 1232 includes, in the illustrated embodiment, first and second wing-coupling members 1236, 1238. Each wing-coupling member 1236, 1238 includes a respective slot 1240, 1242. The slots 1240, 1242 are adapted to receive corresponding wings 1244, 1246 of a lip retracting device 1248.

When the lip retracting device 1248 is worn by a dental procedure subject, insertion of the wings 1244, 1246 into the slots 1240, 1242 serves to stabilize a spatial relationship between the subject and the one or more x-ray sources.

According to one embodiment of the invention, as illustrated, the lip retracting device 1248 includes first and second bite members 1250, 1252. Each bite member 1250, 1252 has a respective one or more film support clips 1253 adapted to support a respective x-ray film package.

In operation, at least one x-ray film package is coupled to the one or more film support clips 1253. The x-ray film package, as is known in the art, includes a sheet of chemical x-ray film enclosed in a light-tight package. The lip retracting device 1248 is coupled to a dental x-ray subject by placing the lips of the patient into the lip-receiving channels of the lip retracting device 1248. The subject then bites down on the bite members to further secure the lip retracting device in a stable spatial relationship to the teeth of the subject. By inserting the wings 1244, 1246 into slots 1240, 1242, the lip retracting device 1248 is stabilized with respect to the dental support structure 100. This serves to stabilize the teeth of the subject and the x-ray film package 1256 with respect to the floor, and thus with respect to an x-ray source. Consequently, the well-known tendency of x-ray subjects to move during exposure of the x-ray film with a resulting non-uniformity of film exposure is reduced.

FIG. 30b shows a further embodiment of the invention including a dental support structure 100 that is adapted to support a dental imaging fixturing system 1230.

Unlike the embodiment of FIG. 30a, the FIG. 30b embodiment includes electronic x-ray sensors 1280, 1282 coupled to the bite members 1250, 1252 respectively. Detecting and imaging x-rays with an electronic image sensor may be used instead of chemical film because electronic image sensors tend to be more sensitive than chemical film, no chemical developing process is required, and the digital images produced by most electronic image sensors are immediately ready for digital manipulation.

In one embodiment of the invention, electronic image sensors each include a respective signal cable with a removable plug.

As discussed above, this spatially stabilized relationship between a subject and the support structure of the invention is found in other embodiments of the invention and in relation to various apparatus and processes.

The use of light guides of the present invention may also promote less air circulation between the patient's mouth and the ambient surroundings. With less air circulation inside the mouth, there may be less evaporation of any treatment composition or whitening composition, which may lead to less dehydration of the mouth. Without wishing to be bound by a theory, it is surmised that since dehydration may lead to increased sensitivity, less dehydration of the mouth may lead to decreased dehydration of the teeth and thus decreased teeth sensitivity during and after treatment. Thus, the use of a light guide during, for example, a bleaching process may potentially be advantageous.

Although the exemplified embodiments of the invention have been described with some specificity, the description and drawings set forth herein are not intended to be limiting, and persons of ordinary skill in the art will understand that various modifications may be made to the embodiments discussed herein without departing from the scope of the invention, and all such changes and modifications are intended to be encompassed within the appended claims. Various changes to the lip retracting device may be made including manufacturing the dimensions differently, using different materials, making the lip retracting device opaque, semi-opaque, transparent, colored, having a textured finish, etc. For example, instead of making the lip retracting device by a single step injection molding, the lip retracting device can be made by welding the various components together, and using multiple molding steps. Also, the four channel retainers can be arranged such that two of the retainers cup two ends of the upper lip and two of the other retainers cup two ends of the lower lip. This alternative lip retracting device can also be incorporated with or without a tongue retainer. Accordingly, many alterations and modifications may be made by those having ordinary skill in the art without deviating from the spirit and scope of the invention.

1. A retracting device for retracting at least a portion of a user's mouth comprising formations adapted for repeatably positioning at least a portion of a subject's mouth with respect to a dental system.

2. The retracting device of claim 1 wherein said dental system comprises a light system, a light guide, an imaging system, a dental treatment composition, an apparatus adapted for aspiration, or combinations thereof.

3. The retracting device of claim 1 wherein said formations comprises inter-engaging, non-inter-engaging or combinations thereof, with other dental tools or apparatus.

4. The retracting device of claim 1 wherein the device comprises at least two channel retainers or flanges, each comprising a race, an inside side wall, an outside side wall, and at least one resilient member attached to the inside side wall of two adjacent channel retainers.

5. The retracting device of claim 1 wherein said formations comprise wing-like members.

6. The retracting device of claim 5 wherein each of said wing-like members is adapted to fit into a formation in an apparatus comprising a an output port, a light guide, a light source, an imaging device or an examination device.

7. The retracting device of claim 3 wherein at east one of said resilient members is integrally molded to the inside side wall of the two adjacent channel retainers.

8. The retracting device of claim 3 wherein at least one of said resilient members comprises two wire-like portions connected by a pad.

9. The retracting device of claim 3 wherein the retracting device comprises at least two arches.

10. The retracting device of claim 3 wherein said retracting device comprises at least two pads attached or molded to the resilient member.

11. The retracting device of claim 4 further comprising a tongue retainer, wherein the tongue retainer is molded or attached to at least one of the channel retainers.

12. The retracting device of claim 1 wherein said device comprises at least four channel retainers or flanges, each comprising a race, an inside side wall, and an outside side wall, and four resilient members, each being integrally molded or attached to two outside side walls of two adjacent channel retainers.

13. The retracting device of claim 12 wherein said formations comprise wing-like members.

14. The retracting device of claim 12 further comprising a tongue retainer attached to two of the channel retainers by at least one secondary resilient member.

15. The retracting device of claim 1 wherein said formation comprises a u-shaped channel configured to accommodate at least one set of a subject's teeth.

16. The retracting device of claim 15 wherein said at least one u-shaped channel supports said at least one channel retainers in substantially fixed spatial relation with respect to one another.

17. The retracting device of claim 4 wherein said wing-like members are adapted to fit into at least one formation in a light guide comprising a second formation adapted to be fitted into a light system or a light system comprising a plurality of light sources.

18. The retracting device of claim 13 further comprising at least at wing-like member for aligning to a mating formation on a light source, an output port, a light guide, an imaging device or an examination cone.

19. The retracting device of claim 1 wherein said formations are adapted for accommodating an imaging film or sensor during imaging.

20. The retracting device of claim 1 wherein at least one of said formations comprises a passively held portion for anchoring the portion to a subject during dental imaging.

21. The retracting device of claim 3 comprising a first alignment formation coupled to a passively held portion wherein the first alignment formation provides alignment to at least one dental feature.

22. The retracting device of claim 21 further comprises a second alignment formation coupled to a formation of an imaging device in a substantially fixed position with respect to the at least one dental feature.

23. The retracting device of claim 3 wherein at least one of said formations comprises a film holder coupled to a passively held portion.

24. The retracting device of claim 23 wherein said film holder is adapted to locate an imaging film, or an imaging sensor, for imaging the at least one dental feature.

25. The retracting device of claim 23 wherein said imaging film, or imaging sensor is integrally formed with the holder.

26. The retracting device of claim 3 wherein at least one of said formations comprises a wing-like member having extended portions.

27. The retracting device of claim 4 wherein at least one of said wings is adapted to be coupled to a target acting as a visual alignment mechanism for the imaging system.

28. The retracting device of claim 26 wherein at least one of said extended portions extends outward and toward the back of the retracting device.

29. The retracting device of claim 26 wherein wing-like members are adapted to enable the imaging system to be aligned on either side of a subject's head.

30. The retracting device of claim 1 wherein at least one of said formation comprises a tab for facilitating insertion and removal of the device.

31. A retracting device comprises at least two channel retainers or flanges, each comprising a race, an inside side wall, an outside side wall, and at least one formation comprising at least one u-shaped channel attached to the inside side wall of two adjacent channel retainers for repeatably positioning a subject's teeth with respect to a treatment composition.

32. The retracting device of claim 31 further comprising formations for positioning the retracting device to a dental system comprising a light system, a light guide an imaging system or combinations thereof.

33. The retracting device of claim 31 wherein said u-shaped channel comprises a dental tray.

34. The retracting device of claim 31 wherein said at least one formation comprises at least two u-shaped channels.

35. A retracting device comprises at least two channel retainers or flanges, each comprising a race, an inside side wall, an outside side wall; at least one resilient member attached to the inside side wall of two adjacent channel retainers; and at least one formation adapted for accommodating an imaging film or sensor during imaging.

36. The retracting device of claim 35 wherein said formation is coupled to a passively held portion adapted for providing alignment to at least one dental feature.

37. The retracting device of claim 36 further comprises an alignment formation coupled to an imaging device in a substantially fixed position with respect to the at least one dental feature.

38. The retracting device of claim 35 wherein at least one of said formations comprises a film holder coupled to a passively held portion.

39. The retracting device of claim 38 wherein said film holder is adapted to locate an imaging film, or an imaging sensor, for imaging the at least one dental feature.

40. The retracting device of claim 38 wherein said imaging film, or imaging sensor is integrally formed with the holder.

41. The retracting device of claim 35 wherein at least one of said formations comprises a wing-like member having extended portions.

42. The retracting device of claim 35 wherein at least one of said wings is adapted to be coupled to a target acting as a visual alignment mechanism for the imaging system.

43. The retracting device of claim 41 wherein at least one of said extended portions extends outward and toward the back of the retracting device.

44. The retracting device of claim 41 wherein wing-like members are adapted to enable the imaging system to be aligned on either side of a subject's head.

45. A retracting device comprises at least four channel retainers or flanges, each comprising a race, an inside side wall, and an outside side wall, and four resilient members, each being integrally molded or attached to two outside side walls of two adjacent channel retainers, said at least one of said resilient members comprises at least one formation adapted for engaging a corresponding formation on an apparatus adapted for aspirating a subject.

46. The retracting device of claim 45 wherein said apparatus comprises an air/gas aspirator or a suction tube.

47. The retracting device of claim 45 wherein said at least one formation of the apparatus comprises a clip.

48. The retracting device of claim 45 wherein said at least one formation on the resilient member comprises a protrusion or a depression.

49. The retracting device of claim 2 wherein said light system comprises at least one heat sink comprising a phase change material.
